(19) United States
(12) Patent Application Publication
(10) Pub. No.: US 2013/0171191 A1
(43) Pub. Date: Jul. 4, 2013

(54) Compositions and Methods for Enhancing Dendritic Cell Resistance to Bacterial Infection and for Enhancing the Mobility of Dendritic Cells for Dendritic Cell-Based Immunotherapy

(71) Applicants: Shigeko Yamashiro-Matsumura, Plainsboro, NJ (US); Fumio Matsumura, Plainsboro, NJ (US)

(72) Inventors: Shigeko Yamashiro-Matsumura, Plainsboro, NJ (US); Fumio Matsumura, Plainsboro, NJ (US)

(73) Assignee: Rutgers, The State University Of New Jersey, New Brunswick, NJ (US)

(21) Appl. No.: 13/341,289

(22) Filed: Dec. 30, 2011

(51) Int. Cl. A61K 39/00; A61K 39/02; A61K 39/12; C12Q 1/02; A61P 31/12; C12N 5/0784; C12N 5/10; A61P 35/00; A61P 31/04

(52) U.S. Cl. 424/204.1; 424/184.1; 424/277.1; 424/234.1; 435/325; 435/29; 435/375

(57) ABSTRACT

Compositions and methods comprising dendritic cells expressing elevated levels of fascin 1 useful in immunotherapy are disclosed.

This application claims priority to U.S. Provisional Application No. 61/248,484 filed Dec. 30, 2011, which is incorporated herein by reference as though set forth in full.

Pursuant to 35 U.S.C. §202(c), it is acknowledged that the U.S. Government has rights in the invention described, which was made in part with funds from the National Institutes of Health, Grant Number RO1CA42742.

### FIELD OF THE INVENTION

This invention relates to the fields of medicine and dendritic cell based therapies. More specifically the invention provides compositions and methods useful for increasing the efficacy of dendritic cell based immunotherapies for the treatment of infection and cancer.

### BACKGROUND OF THE INVENTION

Several publications and patent documents are cited throughout the specification in order to describe the state of the art to which this invention pertains. Each of these citations is incorporated herein by reference as though set forth in full.

Upon maturation, dendritic cells (DCs) change their functions from antigen sampling to antigen presentation, in a process involving massive alterations in their morphology and motility. The actin cytoskeleton plays key roles in multiple aspects of DC function (1-5). For example, antigen presentation by mature DCs depends on the integrity of the actin cytoskeleton (5). Rac1/2, a small G-protein that is responsible for ruffling movements, has been shown to be essential for the interaction of DCs with T-cells (1). WASP (Wiskott-Aldrich Syndrome protein), a molecule that controls Arp2/3-dependent actin polymerization, is required for the formation of the immunological synapse (IS) (2, 6). Although there is abundant evidence that alterations in actin dynamics are important for maturation-associated changes in DCs, it is not clear which actin regulatory proteins induce the profound alterations in morphology and motility observed upon DC maturation. Rac and Cdc42, which control the assembly of membrane ruffling and filopodia, respectively (7), are unlikely to play major roles in these alterations. Rac activity appears to be either unchanged or increased minimally upon maturation (8, 9), while Cdc42 activity is decreased upon maturation (9).

In addition to the profound alterations in morphology and motility, DCs show reduced adhesion to the substrate upon maturation, as evidenced by the loss of podosomes, specialized structures for cell-matrix adhesion (4, 10-12). Podosomes consist of an actin core surrounded by a characteristic ring structure containing adhesion molecules including vinculin, talin, paxilin and integrin (13, 14). DCs transiently lose podosomes at about 20 min after activation with LPS, then recover podosomes by 2 h. Later, they permanently lose podosomes at about 5-7 hr after activation, concomitant with the generation of characteristic veil-like membrane protrusions (4, 10, 11). While the first and transient loss of podosomes is controlled via pathways involving prostaglandin E2, RhoA and rho-kinase (10, 15), as well as ADAM17 (12), it is not clear what causes the second and permanent loss of podosomes in mature DCs.

Fascin1, an actin-bundling protein (see for review 16), is specifically induced to a great extent upon DC maturation while it is not detectable in immature DCs (17-20). Other blood cells such as primary macrophages and T-cells do not express or induce fascin1. The induction of fascin1 in DCs suggests a specific role for fascin1 in DC maturation. Fascin1 has been suggested to be critical for assembly of filopodia or membrane protrusions. Fascin1 is localized to filopodia (21-25) and fascin1 overexpression induces membrane protrusions and increases cell motility of epithelial cells and colon carcinomas (26-28). Conversely, fascin1 knockdown has been reported to block assembly of filopodia in cultured mammalian cells (27-29). In bone marrow-derived DCs, fascin1 depletion by anti-sense treatment resulted in the inhibition of both dendrite formation and T-cell activation (19, 20, 30). However, little is known about what roles fascin1 plays in motility and cytoskeletal reorganization associated with DC maturation.

### SUMMARY OF THE INVENTION

The present inventors have discovered that elevated fascin 1 expression levels are associated with enhanced migration of dendritic cells into lymph nodes for antigen presentation to T cells. Accordingly such cells have utility for use in immunotherapy for the treatment of cancer and/or viral and bacterial infections. Thus in one aspect, the invention provides an isolated dendritic cell expressing elevated levels of fascin 1, which exhibits accelerated migration. In one embodiment elevated fascin 1 expression levels are obtained via introduction of an expression vector which comprises a nucleic acid encoding fascin 1 which is operably linked to a constitutive or inducible promoter. Alternatively, culture conditions for dendritic cells can be set up which facilitate isolation and selection of fascin 1 over-expressing cells. Such cells and compositions comprising the same have utility in the methods of the invention.

In another aspect of the invention, a method for the treatment of cancer in a patient in need thereof is provided. An exemplary method entails incubating dendritic cells with a tumor antigen of interest, thereby priming dendritic cells, and then selecting dendritic cells expressing elevated levels of fascin 1. The selected dendritic cells are reinfused into said patient, thereby provoking a cytotoxic T cell response to cancer cells expressing the antigen of interest, the cytotoxic response causing a reduction in tumor cell burden. The method can also comprise the administration of a chemotherapeutic agent.

In yet another aspect of the invention, a method for the treatment of bacterial or viral infection in a patient in need thereof is provided. An exemplary method entails incubating dendritic cells with a bacterial or viral antigen of interest, thereby priming said dendritic cells and selecting dendritic cells expressing elevated levels of fascin 1. The selected dendritic cells are reinfused into said patient, thereby provoking a cytotoxic T cell response to cells expressing the bacterial or viral antigen of interest, the cytotoxic response causing a reduction in infected cell burden. The method can also comprise the administration of an antibiotic or antiviral agent.

Listeria monocytogenes infection makes vacuoles (including bacteria encapsulated phagosomes and autophagosomes) of wild type DCs more acidic when compared to fascin1 KO mice indicating that fascin 1 accelerates lysosomal fusion with phagosomes or autophagosomes thereby killing intracellular bacteria. Thus, the invention also provides methods for identifying agents which modulate the actions of fascin1 mediated DC resistance to Listeria monocytogenes.

### BRIEF DESCRIPTION OF THE DRAWINGS

FIG. 1. Characterization of wild type and fascin1 null DCs. A, FACS analyses of wild-type (red line) and fascin1-deficient (blue line), mature DCs. Black lines, controls without antibody labeling. a, CD11c; b, MHC-II; c, CD86; d, fascin1. e & f, FACS analyses of wild type (e) and KO (f) DCs double-labeled with CD86 and fascin1 antibodies. B, Western blot analyses. Immature (1M, lanes 1 and 3) and mature (M, lanes 2 & 4) DCs prepared from wild type (lanes 1 & 2) and fascin1 KO (lanes 3 & 4) mice were analyzed with the indicated antibodies. C, Immunofluorescent localization of fascin1 and CD86 in mature wild-type (a-c) and fascin1 KO (d-f) DCs. Mature DCs were double labeled with anti-CD86 (a and d, green) and mouse anti-fascin1 (b and e, red) antibodies. c & f, merged images. Bar, 10 mm. D, Localization of fascin1 and F-actin in wild type (a-f) and fascin1 KO (g-l) DCs. Cells were labeled with chick anti-fascin1 (a, d, g & j, green) and rhodamine phalloidin (b, e, h & k, red). Images were taken at the ventral surface (a-c & g-i), as well as at the middle (d-f & j-l) of the same cells. c, f, i, & l, merged images. Arrowheads indicate co-localization of fascin1 and F-actin at the cortex of veil-like protrusions. Bar, 10 mm.

FIG. 2. Fascin1 is critical for membrane dynamics. A-C, Time-lapse, phase-contrast microscopy was performed with wild type (A) and fascin1-null (B, C) DCs that had been plated on glass coverslips. B shows a fascin1-null DC with a spread shape, the dominant phenotype; C shows a cell with a rounded shape, a less common phenotype. Numbers are in sec. Bar, 5 mm. D-F, kymographs generated from the white one-pixel lines at time 0 in A-C, respectively. Dashed lines in D-F indicate the boundary of the membrane protrusions. G & H, box plot analyses of protrusion (G) and retraction (H) rates calculated from kymographs. The bottom and top of each box indicate the first and third quartiles, respectively, and dots indicate outliers. Four asterisks, p<0.0001. I & J, Phase-contrast, time-lapse images of fascin1-null mature DCs exogenously expressing GFP (I) or GFP-fascin1 (J). The last images show fluorescent images at the end (20 min) of imaging to confirm GFP-expression. Numbers are in sec. Bar, 5 mm. K & L, kymographs of GFP (K)-, and GFP-fascin1 (L)-expressing DCs generated from the time-lapse images in I and J, respectively. M & N, box plot analyses of protrusion (M) and retraction (N) rates of fascin1-null DCs exogenously expressing GFP or GFP-fascin1.

FIG. 3. Fascin1 is critical for chemotaxis and Langerhans cell migration into draining lymph nodes. A, In vitro chemotaxis toward the chemokine CCL19, measured with a modified Boyden chamber. (p=0.0095). B, Immunofluorescence imaging of Langerhans cells in ear epidermal sheets from wild type (a & c) and fascin1 null (b & d) mice without (control, a & b) and with FITC painting (+allergen, c & d). Langerhans cells (indicated by arrowheads) were labeled with the MHC-II antibody. Representative images from four independent experiments. C, Box plot analyses of Langerhans cell distribution without or with allergen treatment. NS, no statistical significance; one asterisk, p<0.05; two asterisks, p<0.01, three asterisks, p<0.001; four asterisks, p<0.0001. D, Box plot analysis of FITC-bearing DCs migrated into draining lymph nodes. P=0.0059.

FIG. 4. Morphological characterization of wild type and fascin1 null DCs. A, Scanning electron micrographs of wild-type (a) and fascin1-deficient (b) mature DCs. Bar, 10 mm. B, Orthogonal views of wild-type (a) and fascin1-deficient (b), CD86^{high} DCs. Mature DCs were double stained with phalloidin and anti-CD86 antibody. Only phalloidin staining is shown here. The xy images are on the ventral surface. Both xz and yz images are shown with the top and bottom of cells indicated by dashed lines 5 mm. C, D and E, Box blot analyses of thickness (C), area (D) and circularity (E) of wild-type and fascin1-deficient DCs. Thickness was determined without (w/o cfg) or with (w. cfg) cytospin.

FIG. 5. Fascin1 expression and actin-bundling activity is critical for podosome disassembly of mature DCs. A, Immunofluorescence of immature (a & b) and mature (c & d) DCs from wild-type (a & c) and fascin1 null (b & d) mice labeled with anti-vinculin (red) and anti-CD11c (green) antibodies. Arrows, podosomes with the characteristic ring structure; arrowheads, focal adhesions. Bar, 15 mm. B, Mature fascin1^{high} DCs (arrow) show no podosome assembly. Bar, 10 mm. C, Statistical analyses of podosome assembly. CD11c-positive immature (blue bar) and mature (red) DCs with at least five vinculin-positive podosomes, were judged as DCs with podosomes. Wild-type, mature DCs with very high fascin1 expression (more than 10 times higher than background, pink bar) were also examined for podosome assembly. D, Effects of forced expression of GFP control (a-c), GFP-Wild-type-fascin1 (d-f), GFP-A-fascin1 (g-i) and GFP-D-fascin1 (j-l) on podosome assembly. Fascin1 null DCs were transfected with GFP control (a-c), as well as with wild type and fascin1 mutants, and stained with anti-vinculin antibody (red, b, e, h, k). GFP signal (green, a, d, g, j); merged images (c, f, i, l). Arrowheads in j-l show podosomes. Bar, 10 mm. E, Statistical analyses of podosome loss. Podosomes were counted in DCs exogenously expressing control GFP, GFP-W-fascin1, GFP-A-fascin or GFP-D-fascin, and cells were categorized as having less than 4 podosomes or more than 5 podosomes.

FIG. 6. Fascin1 is localized to podosomes in THP-1 cells and microinjection of fascin1 induces podosome disassembly. A, Co-localization of fascin1 and a-actinin at podosomes. THP-1 cells differentiated with TPA were labeled with anti-fascin1 (b & e, green) and anti-a-actinin (red, a & d) antibodies. c & f, merged images. Images in d-f show enlargements of the boxed areas in a-c. Arrowheads, podosomes. B, Disassembly of podosomes by microinjection of fascin1. THP-1 cells were microinjected with FITC-labeled BSA (a) or GFP-fascin1 (c) and counter-stained with phalloidin (b & d). Arrows indicate injected cells while arrowheads indicate podosomes. C, Percentage of cells with podosome arrays of un-injected cells (green); or after microinjection of fascin1 (red) or FITC-BSA (blue). Representative data from three independent experiments is shown. Approximately 20 injected cells for each condition were counted for each set of experiments.

FIG. 7. Higher susceptibility of fascin 1 KO DCs to Lm. A. Lm replication during the initial 4 hour of infection. B. More wild type DCs can survive 24-48 hr post infection.

FIG. 8. A-D Immunofluorescence of wild type DCs infected with Lm for 3 hour (A and B) and 24 hour (C and D) Lm (red) fascin 1 (green). E and F show Fascin 1 KO DCs infected with Lm 24 hours post infection. Lm (red), CD86 (green). G. Quantitative analyses of Lm infection in fascin1-positive (red bars) wild type DCs, fascin 1-negative (green) wild type Csc and fascin1 KO DCs (blue). 24 hour post infection. Number of infected bacteria per DC were quantified and percentiles shown.

FIG. 9. Higher vacuolar acidification in wild type DCs. A, Phagosomal pH measure with CFMDA/Rhodamine-labled Lm. B, Vacuolar pH measure by a LysoSensor probe.

FIG. 10. Wild type DCs show higher autophagy flux than do fascin1 KO DCs. Autophagolysosomal fusion of tfLC3 turned yellow signals to red.

### DETAILED DESCRIPTION OF THE INVENTION

Dendritic cells (DCs) play central roles in innate and adaptive immunity. Upon maturation, DCs assemble numerous veil-like membrane protrusions, disassemble podosomes, and travel from the peripheral tissues to lymph nodes to present antigens to T-cells. These alterations in morphology and motility are closely linked to the primary function of DCs, antigen presentation. However, it is unclear how and what cytoskeletal proteins control maturation-associated alterations, in particular, the change in cell migration.

Fascin1, an actin-bundling protein, is specifically and greatly induced upon maturation, suggesting a unique role for fascin1 in mature DCs. To determine the physiological roles of fascin1, we characterized bone marrow-derived, mature DCs from fascin1 knockout mice. We found that fascin1 is critical for cell migration: Fascin1 null DCs exhibit severely decreased membrane protrusive activity. Importantly, fascin1 null DCs have lower chemotactic activity toward CCL19 (a chemokine for mature DCs) in vitro, and in vivo, Langerhans cells show reduced emigration into draining lymph nodes. Morphologically, fascin1 null mature DCs are flatter and fail to disassemble podosomes, a specialized structure for cell-matrix adhesion. Expression of exogenous fascin1 in fascin1 null DCs rescues the defects in membrane protrusive activity, as well as in podosome disassembly. These results indicate that fascin1 positively regulates migration of mature DCs into lymph nodes, likely by increasing dynamics of membrane protrusions, as well as by disassembling podosomes.

Dendritic Cell (DC) therapy represents a new and promising immunotherapeutic approach for treatment of advance cancer as well as for secondary prevention of cancer. For decades, cancer researchers have been interested in immunologic treatment using vaccines against cancer but with little progress. However, recent advances in recognition of the importance of 1) tumor associated antigens that can be used to vaccinate patients, and 2) the dendritic cell as a potent blood cell to present such antigens and stimulate the naïve immune system together leads to successful implementation of Dendritic Cell therapy with reports of complete responses even in stage 1V cancer patients who have failed all other therapies.

Dendritic Cell (DC) Therapy or so-called Dendritic Cell vaccine is a newly emerging and potent form of immune therapy used to treat cancer, AIDS and other serious conditions. In case of cancer, dendritic cell therapy is an immune therapy which harnesses the body's own immune system to fight cancer. The dendritic cell itself is an immune cell whose role is the recognition, processing and presentation of foreign antigens to the T-cells in the effector arm of the immune system. Although dendritic cells are potent cells, they are not usually present in adequate quantity to allow for a potent immune response. Dendritic cell therapy thus involves the harvesting of blood cells (i.e., monocytes or macrophages) from a patient and processing them in the laboratory to produce dendritic cells which are then given back to a patient in order to allow massive dendritic participation in optimally activating the immune system. The present invention provides compositions and methods for increasing the efficacy of tumor antigen presentation for example, thereby improving upon or augmenting available therapies for the treatment of cancer. The invention also provided compositions and method for treating viral and bacterial infections.

### DEFINITIONS

“Fascin 1” is an actin-bundling protein that contributes to the architecture and function of cell protrusions and microfilaments in cell adhesion, interactions and motility. The sequence of human fascin 1 encoding nucleic acids and protein is available in GenBank, Accession No. NM_{—}003088.2.

The phrase “dendritic cell vaccine” refers to a process where dentritic cells are either extracted from the patient to be treated or from a genetically compatible donor and then exposed to, or “primed” with an antigen of interest, e.g., a tumor antigen from the patients tumor or a viral antigen. This combination of dendritic cells and antigens is then injected into the patient, where the dendritic cells will work to program the T cells, thereby inducing a cytotoxic response to the cells expressing the priming antigen.

A “therapeutically effective amount” of a compound or a pharmaceutical composition refers to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Cancers that may be treated using the present protocol include, but are not limited to: cancers of the prostate, colorectum, pancreas, cervix, stomach, endometrium, brain, liver, bladder, ovary, testis, head, neck, skin (including melanoma and basal carcinoma), mesothelial lining, white blood cell (including lymphoma and leukemia) esophagus, breast, muscle, connective tissue, lung (including small-cell lung carcinoma and non-small-cell carcinoma), adrenal gland, thyroid, kidney, or bone; glioblastoma, mesothelioma, renal cell carcinoma, gastric carcinoma, sarcoma, choriocarcinoma, cutaneous basocellular carcinoma, and testicular seminoma.

The dendritic cell vaccine may be administered in conjunction with other medications typically used to treat cancer or viral infection. Suitable chemotherapeutic agents include, but are not limited to: toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In a particular embodiment, the chemotherapeutic agent is selected from the group consisting of: placitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

“Concurrently” means (1) simultaneously in time, or (2) at different times during the course of a common treatment schedule.

“Sequentially” refers to the administration of one component of the method followed by administration of the other component. After administration of one component, the next component can be administered substantially immediately after the first component, or the next component can be administered after an effective time period after the first component; the effective time period is the amount of time given for realization of maximum benefit from the administration of the first component.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The terms “transfected” and “transfection” as used herein refer to methods of delivering exogenous DNA into a cell, particularly a dendritic cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

### Methods for Increasing Expression of Fascin1 in DCs

The present inventors have discovered that fascin1 plays a key role in immunity, antigen presentation and resistance to certain bacterial infections. Accordingly, the provision of DCs expressing elevated levels of fascin1 for autologous reinfusion for therapeutic purposes is highly desirable. At least two approaches could be taken to increase fascin1 expression in a target cell population. In one approach, DCs would be harvested from the patient, propagated in culture to obtain large numbers and exposed to an antigen of interest. Dendritic cells would then be subjected to FACS using cell surface markers including CD83 and CD86 as expression levels of these surface markers have been previously correlated with high fascin1 expression. FACS would allow us to isolate and purify DCs expressing high levels of fascin 1 from the mixed population. These selected dendritic cells with high fascin1 expression could be reinfused into the patient in order to provoke a cytotoxic T cell response to the antigen of interest.

In another approach, DCs will be isolated and heterologous nucleic acids encoding GFP-tagged fascin1 would be introduced into the cells optionally under the control of a DC specific promoter and enhancer thereby creating a transfected cell population that exhibits increased expression of fascin 1 after antigen exposure. Vectors for this purpose are commercially available and include without limitation AAV vectors, lentiviral vectors, and plasmid vectors. The sequence information for fascin1, as well as it promoter and enhancer sequences, has been deposited in Genbank thereby facilitating the creation of such fascin1 expressing vectors. Transfected dendritic cells would be exposed to an antigen of interest. Cells expressing the desired levels of fascin1 can then be identified and sorted by FACS using GFP signals. Once sufficient numbers are obtained, the cells are then reinfused into the patient as described above.

The following materials and methods are provided to facilitate the practice of the present invention.

### Reagents, Antibodies and Fascin1-Deficient Mice:

The following antibodies were used: FITC-conjugated hamster anti-mouse CD11c monoclonal (BD Biosciences, San Diego, Calif.), FITC-conjugated rat anti-mouse CD86 (B7-2) monoclonal, FITC-conjugated rat anti-mouse I-A/1-E (MHC-II) monoclonal (BD Biosciences), mouse anti-vinculin monoclonal (Sigma, St. Louis Mo.), rabbit anti-a-actinin antibody (25), mouse anti-fascin mouse monoclonal (55k-2) (25), chick anti-fascin antibody (generated by Ayes Labs (Tigard, Oreg.) using recombinant human fascin1 as an antigen). GM-CSF and CCL19 (MIP3b) were purchased from Invitrogen (Camarillo, Calif.)

Fascin1 KO heterozygous mice were generated by Lexicon Pharmaceuticals, Inc (Woodlands, Tex.) from an ES cell line (OST124903) (Lexicon's OmniBank® library of gene KO ES cell clones), and backcrossed with C57/BL6 female mice for more than 16 generations (31). Both RT-PCR and Western blot analyses revealed that the loss of fascin1 is not compensated with the expression of the fascin1 paralogues (such as retina fascin2 and testis fascin3) (31). For each experiment with homozygous mice, their wild-type littermates were used as a control. All experimental procedures and protocols for mice are approved by the Animal Care and Facilities Committee at Rutgers. Mice were housed in an AAALAC-accredited animal facility at Rutgers.

### Preparation of Bone Marrow DCs:

Preparation of mouse bone marrow DCs was according to the method described in Inaba et al (32) with slight modification. Briefly, single cell suspension was prepared from bone marrow of femurs and tibias, and plated on 65 mm dishes in DMEM containing 10% fetal calf serum and 10 ng/ml of GM-CSF for 7-10 days. Non-adherent cells were collected and DCs were purified by centrifugation over a 13.7% (w/v) metrizamide discontinuous gradient. More than 85% of cells collected at the interface of the gradient were positive for CD11c. Cells were matured by overnight culture in the presence of 100 ng/ml of lipopolysaccharide (LPS, Sigma).

### FACS Analyses

Mature DCs were fixed with methanol or formalin, and stained with FITC-labeled anti-DC markers including CD86, CD11c, and MHC-II. For double labeling, methanol-fixed cells were blocked with a rat anti-mouse CD16/CD32 antibody (mouse Fc Block, BD Pharmingen), incubated with the mouse anti-fascin1 antibody (clone 55k-2) together with the FITC-labeled CD86 antibody, and then the fascin antibody was labeled with a R-PE-labeled goat anti-mouse IgG. Flow cytometry was performed with a Coulter Cytomics FC500 flow cytometer.

### Immunofluorescent Microscopy and Measurements of Thickness, Area and Circularity

For staining with antibodies against CD11c, CD86, MHC-II, and vinculin, as well as for staining with rhodamine phalloidin (Molecular Probes, Eugene, Oreg.), DCs were fixed with 3.7% formaldehyde, and permeabilized with 0.2% Triton X-100 or 100% acetone. Absolute methanol fixation at −20° C. was used for double labeling with the anti-fascin1 mouse monoclonal (clone 55k-2) and the anti-CD86 antibody, and for double staining with anti-fascin1 and anti-a-actinin antibodies. Images were taken as Z-stacks (0.2 mm spacing) with a DeltaVision Image Restoration Microscope system (Applied Precision Instrument, LLC Issaquah, Wash.), deconvolved either with the softWoRx software (Applied Precision Instruments) or the Huygens software (Scientific Volume Imaging, Hilversum, Netherlands). Projected images were generated with SoftWoRx or ImageJ (http://rsb.info.nih.gov/ij/). In some experiments, images were taken on a Nikon TE300 microscope with a 60× objective lens (NA 1.4). Exposure times for imaging and settings for deconvolution were constant for all samples to be compared within any given experiment. For presentation, image contrast and brightness were adjusted with Photoshop (Adobe, San Jose, Calif.).

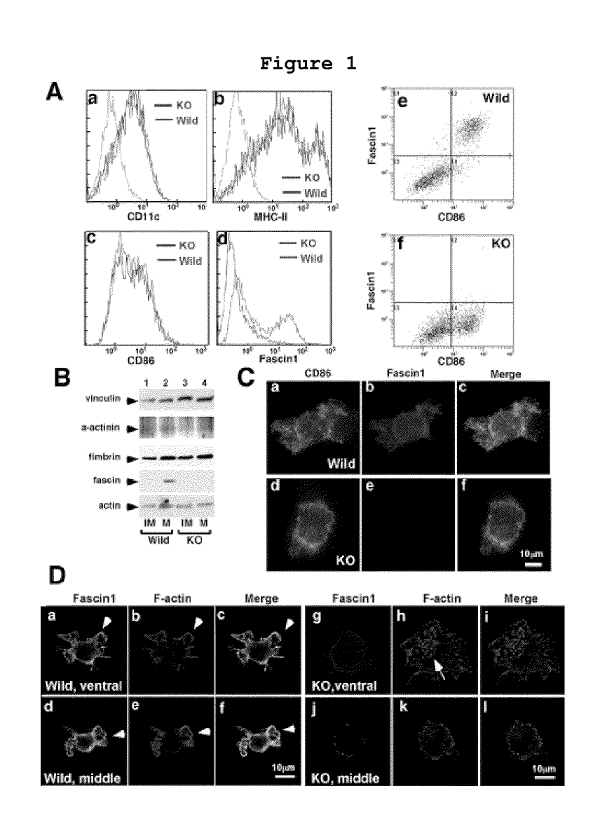

For measurements of thickness, area and circularity, wild type and fascin1 KO DCs were labeled with the FITC-labeled CD86 antibody, rhodamine phalloidin and DAPI. Because the expression of CD86 is well correlated with that of fascin1 (see FACS analyses shown in FIG. 1A), CD86^{high} DCs were chosen to compare differences in thickness, area and circularity between fascin1-expressing wild type and fascin1 null DCs. Orthogonal images created by SoftWoRx were used for measurement of thickness. Areas were measured with xy images of DCs at the ventral focal plane and circularities were measured with Z-projected images. Both areas and circularities were measured using ImageJ software.

### Live Cell Imaging, Kymography, Microinjection and Transfection

For phase-contrast, live cell imaging, DCs were placed at 37° C. in a temperature controlled incubator (MS200D, Narishige) and observed under a Nikon microscope (TE300) with a 40× Plan Fluor phase-contrast (NA 0.60) objective lens. Time-lapse images were taken every 10 sec for 20-30 min by a CCD camera (CoolSnap-fx, Roper Scientific) with IPLab image analysis software (Scanalytics). Two to three kymographs were generated for each cell with randomly chosen, one-pixel lines using ImageJ (NIH) with the Kymograph plug-in (written by J. Rietdorf and A. Seitz, EMBL). Kymographs were then analyzed using ImageJ to determine rates of membrane protrusions and retractions.

Microinjection of GFP-fascin1 into differentiated THP-1 (human acute monocytic leukemia cell line) cells was performed as follows: Cells were first differentiated into macrophages by the treatment of 200 nM of 2-O-Tetradecanoylphorbol-13-acetate (TPA) for overnight as described (33). Microinjection was performed as described previously (26) using GFP-fascin1 at a needle concentration of 9 mg/ml. As a control, FITC-labeled BSA was injected. After 1 hr incubation, cells were fixed with formaldehyde, permeabilized with acetone, and counterstained with rhodamine-labeled phalloidin or the anti-vinculin antibody to determine effects on podosome assembly. To estimate levels of fascin1 in injected cells, injected cells were stained with the fascin1 antibody, and fluorescent intensities were compared with those of un-injected cells and fascin1-expressing DCs. It was found that injection increased the fascin1 level five to ten times over the level of endogenous fascin1 in THP-1 cells, the level of which is comparable to that found in wild type DCs.

Mature or immature DCs were transfected with a human GFP-fascin1 fusion construct (26) using an Amaxa Nucleofector II according to the manufacture's instructions.

### In Vitro Assay for Chemotaxis

Chemotaxis of mature DCs was assayed in triplicate using a modified Boyden chamber assay. CCL19 (MIP3b), a chemokine for mature DCs, was added in bottom wells at the concentration of 0.6 mg/ml. Mature wild type and fascin1 KO DCs (2×10⁵ cells) were placed on top wells of a collagen-coated Boyden chamber with 3 mm hole (Corning, Lowell, Mass.). After 24 hr incubation, cells transmigrated into the bottom chamber were counted.

Preparation of Epidermal Sheets and Assay for DC Migration into Lymph Nodes

Epidermal sheets before and after stimulation by FITC painting were prepared essentially as described (34). Briefly, the dorsal surfaces of both ears of wild type and fascin1 KO mice were painted with 25 ml of 1% FITC in acetone:dibutylphthalate (1:1). After 24 hr, epidermal sheets were isolated from dorsal halves of the ears and stained with anti-MHC-II antibody followed by Cy3-labeled donkey anti-rat antibody. Fifteen to twenty randomly selected fields were photographed with a Nikon TE300 with a 20× objective and the number of Langerhans cells per field (area, 149,000 mm²) were counted.

Langerhans cells migrated into draining lymph nodes were prepared essentially as described (35, 36). Briefly, mice were painted with the FITC solution as described above except that both dorsal and ventral sides of ears were painted with 15 ml of solution (total 30 ml per one ear). After 24 hr, draining lymph nodes (auricular) were excised and cell suspension was prepared through cell strainers (Falcon, 70 mm). DCs were then enriched on metrizamide discontinuous gradients as described above and cytospun onto coverslips. FITC-bearing DCs were identified and counted by fluorescence and phase-contrast microscopy, and Langerhans cell migration was expressed as numbers of FITC-bearing DCs per lymph node.

### Scanning Electron Microscopy

DCs grown on coverslips were fixed with 2% glutaraldehyde in 0.1 M sodium cacodylate, pH 7.3 for 20 min at room temperature, and dehydrated by submerging graded ethanol solutions. After critical point drying and platinum coating, images were taken with an Amray 19301 scanning microscope.

### Statistical Analysis.

Statistical analyses were performed using Student's t-test (http://www.physics.csbsju.edu/stats/t-test.html).

The following examples are provided to illustrate certain embodiments of the invention. They are not intended to limit the invention in any way.

### Example 1

### Fascin 1 Promotes Cell Migration of Mature Dendritic Cells

To determine the physiological function of fascin1 in DC maturation, we generated fascin1 knockout (KO) mice (31). Fascin1 KO mice provide an excellent experimental system, allowing us to analyze fascin function in DCs with little experimental manipulation. In addition, fascin1 KO mice provide fascin1 null phenotypes that are complete and uniform, unlike anti-sense or siRNA approaches that tend to be partial and variable. Our results indicate that fascin1 profoundly alters DC cytoskeletal organization and plays critical roles in migration of mature DCs into lymph nodes for antigen presentation.

Fascin1-Deficiency does not Alter Expression of DC Maturation Markers or Other Actin-Binding Proteins.

As a first step toward characterization of fascin1 null DCs, FACS analyses were performed to determine whether fascin1 deficiency affects expression of DC markers including CD11c, CD86 and MHC-II when bone marrow-derived DCs were activated by 100 ng/ml of lipopolysaccharide (LPS). As shown in FIG. 1A, surface expression of the DC marker CD11c (a), and the maturation markers MHC-II and CD86 (b and c), were similar between wild type and fascin null, mature DCs, indicating that fascin1 null DCs are able to “mature” in terms of the expression of maturation markers. As expected, fascin1 was expressed only in wild type but not fascin1 KO DCs (d, f). As shown in panel e, double labeling with anti-fascin1 and anti-CD86 antibodies revealed a strong correlation between fascin1 and CD86 expression in wild type DCs. Before LPS activation, both wild type and fascin1 null DCs expressed similar levels of CD11c, while neither cell type expressed appreciable levels of CD86 (data not shown). These results are consistent with previous reports that fascin1 depletion by anti-sense treatments did not alter expression of DC maturation markers (19, 20, 30).

Western blot analyses confirmed that fascin1 is induced upon maturation of wild type DCs (lane 2 of FIG. 1B), while it is absent in immature, wild type DCs (lane 1), as well as immature and mature fascin1 KO DCs (lanes 3 & 4, respectively). We examined whether fascin1 deficiency alters expression of other actin-bundling proteins including fimbrin and a-actinin. Fimbrin levels are largely unaltered between wild type and fascin1 KO DCs, regardless of maturation. a-actinin was barely detectable in DCs, and its levels appear unchanged. The levels of vinculin, however, are slightly increased in fascin1 KO DCs.

FIG. 1C shows immunofluorescence localization of fascin1 and CD86 of wild type, as well as fascin1 KO DCs. As expected, fascin1 KO DCs showed no fascin1 staining while CD86 staining intensities were similar between wild type and fascin1 KO DCs, confirming the FACS analyses. As previously reported (17, 25, 37, 38), fascin1 was localized at filopodia-like structures.

### Fascin1 is Present in the Cortex of Membrane Protrusions.

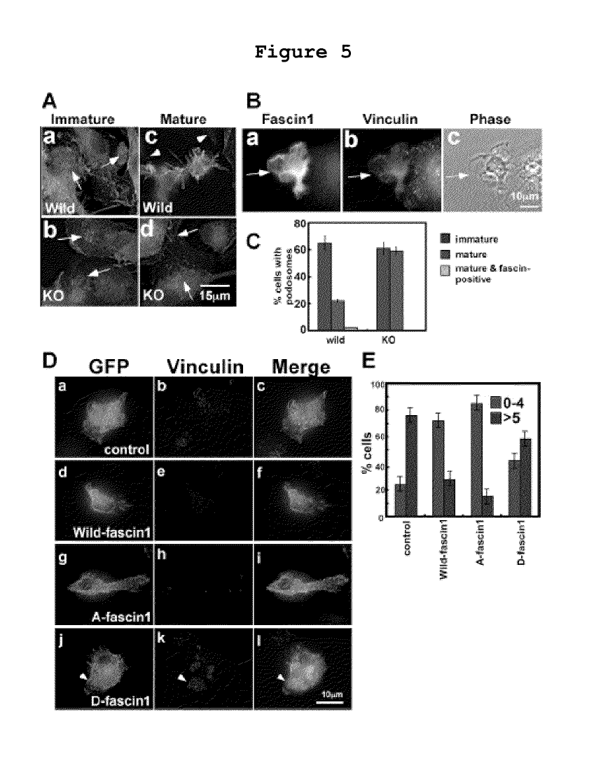

We found that fascin1 was also localized at the cortex of veil-like membranes (a more prominent structure than filopodia in mature DCs) of mature wild-type DCs. FIG. 1D shows wild type and fascin1 KO DCs double-labeled with phalloidin and anti-fascin1 antibody and imaged at two different focal planes (the ventral surface and a middle). In wild type DCs expressing high levels of fascin1, both fascin1 and F-actin were co-localized at the cortex of veil-like protrusions (arrowheads in a-f). In contrast, fascin1 KO DCs were more spread and showed many fewer veil-like protrusions either at the ventral surface or at the middle focal plane. In addition, fascin1 KO DCs frequently showed a cluster of many prominent actin dots at the ventral surface, which were reminiscent of podosomes (arrow in h). In contrast, most wild type DCs did not exhibit such large and clustered dots. As described later, vinculin labeling revealed that these dots observed in fascin1 KO DCs indeed represent podosomes (see FIG. 5).

### Fascin1 is Essential for Dynamics of Membrane Protrusions.

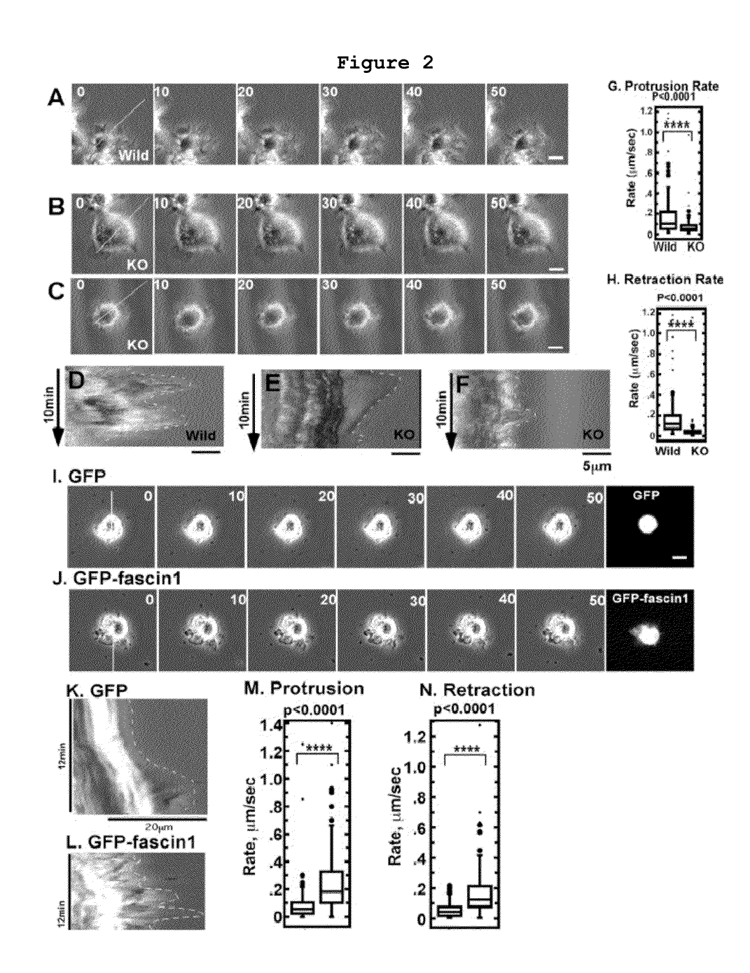

The presence of fascin1 in the cortex of membrane protrusions (FIG. 1D) prompted us to examine whether the dynamics of membrane protrusions is altered by fascin1-deficiency. To this end, we imaged live DCs plated on glass coverslips using phase-contrast microscopy (data not shown). FIG. 2 (A-C) illustrates representative still images of wild type and fascin1 null (B & C, DCs, respectively. FIG. 2B represents the majority of fascin1 null DCs whereas FIG. 2C represents a minor fraction (less than 20%) of fascin1 null DCs. These images clearly demonstrate that membrane activity of wild-type DCs is much more vigorous that that in fascin null DCs. Kymograph analyses (FIGS. 2D-F) confirm that fascin1-null DCs (E & F) displayed greatly diminished dynamics compared to wild-type DCs (D). Box plot analyses of protrusion (G) and retraction (H) rates generated from kymographs (representing 7 different live cell imaging experiments) revealed that the median value of the protrusion rate for wild-type DCs (0.11 mm/sec, n=83) was 2.1 times higher than that of fascin1-deficient DCs (0.053 mm/sec, n=85), and the retraction rate in wild-type DCs (0.10 mm/sec, n=84) was 4 times higher than that of fascin1-deficient DCs (0.025 mm/sec, n=87).

To determine whether fascin1 is responsible for the dynamic membrane movements, we re-expressed GFP-fascin1 in fascin1 KO DCs, and tested whether fascin1 was able to rescue the poor dynamics of membrane protrusions. Phase-contrast, time-lapse microscopy was performed with fascin1 KO DCs expressing GFP alone (control) or GFP-fascin1). As shown in both still images (FIGS. 21 and J) and kymographs (K and L), DCs expressing GFP-fascin1 (J and L) exhibited much greater membrane protrusion dynamics than did DCs expressing control GFP (I and K). Box plot analyses (M and N) revealed a significant difference in both protrusion and retraction rates (p<0.0001): The median protrusion and retraction rates for DCs expressing GFP-fascin1 were 0.18 mm/sec (n=103) and 0.13 mm/sec (n=98), respectively, whereas those of DCs expressing GFP were 0.058 mm/sec (n=74) and 0.043 mm/sec (n=50), respectively. Thus, the protrusion/retraction rates of GFP-fascin1-expressing DCs were comparable to those of wild-type DCs, while those for GFP-expressing DCs were similar to those of fascin1-deficient DCs. These results indicate that fascin1 induction upon maturation is responsible for the vigorous dynamic membrane movements of mature DCs.

### Fascin1 is Important for In Vitro Chemotaxis of Mature DCs Toward CCL19.

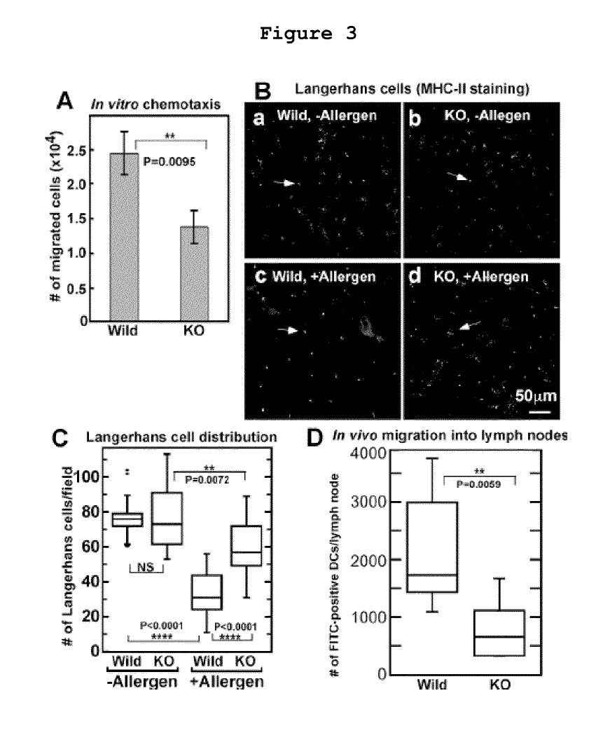

The lower membrane protrusive activity of fascin1-deficient, mature DCs would be predicted to impair their migratory efficiency. To test this idea, we examined, using a modified Boyden chamber (3 mm holes with collagen coating), whether fascin1 deficiency affects chemotaxis of mature DCs toward CCL19 (MIP3b). As FIG. 3A shows, fascin1 deficiency reduced chemotaxis by 42% (p=0.0095).

### Fascin1-Deficient Mice Show Reduced Migration of Langerhans Cells.

Consistent with the impaired chemotaxis of fascin1 KO DCs toward CCL1 in vitro, we found that Langerhans cells of fascin1 KO mice showed reduced migration into lymph nodes. Twenty-four hours after painting of dorsal sides of both ears with an allergen of FITC, epidermal cell sheets were prepared and stained with a MHC-II antibody. FIG. 3B shows representative immunofluorescence images of wild and KO epidermal sheets. Without allergen, both wild type and KO sheets showed a similar Langerhans cell distribution. After stimulation with the allergen, Langerhans cells from wild type mice clearly exhibited decreased cell density when compared to KO. Quantitative data (FIG. 3C) indicate that the mean density of wild-type Langerhans cells after stimulation was about half of that of fascin1 KO (p<0.001) while the density of Langerhans cells before stimulation was statistically similar between wild type and fascin1 KO mice.

To confirm that the above difference in Langerhans cell density is indeed due to migration of Langerhans cells into lymph nodes, we measured the number of FITC-bearing DCs in draining lymph nodes following stimulation by FITC painting for 24 hr. As FIG. 3D shows, there were over twice as many FITC-bearing DCs per lymph node in wild type mice as in fascin1 null mice (p=0.0059). Taken together, these results show that Langerhans cell migration is impaired in fascin1 KO mice, and support our hypothesis that fascin1 plays a critical role in DC migration into lymph nodes by promoting podosome disassembly and increasing dynamics of membrane protrusions.

Fascin1-Deficient DCs are More Spread and Thinner with Fewer Membrane Protrusions.

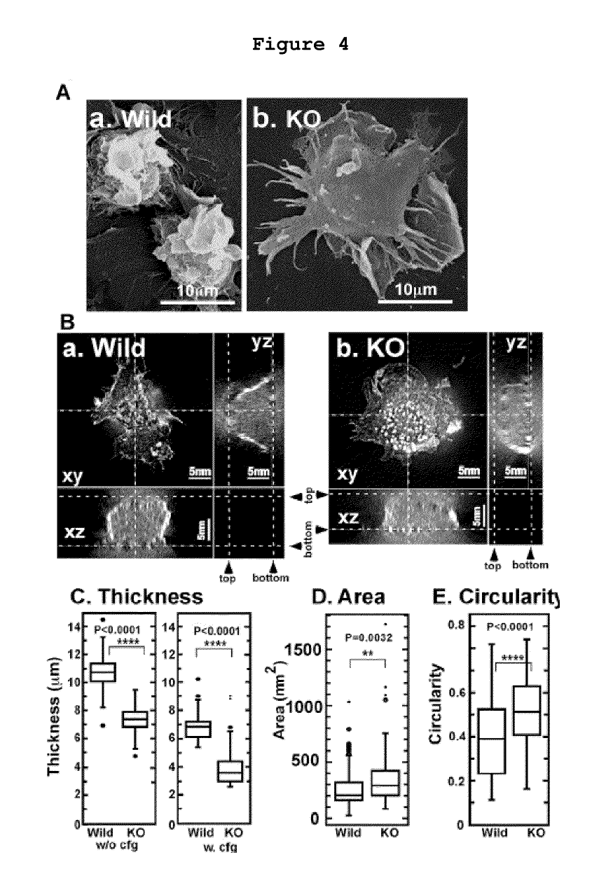

Fascin1-deficient, mature DCs are morphologically very different from their wild-type counterparts. FIG. 4A shows scanning electron microscopy of wild type and fascin1 null mature DCs. Fascin1-deficient DCs were much thinner and more spread with fewer and smaller dorsal ruffles than wild-type DCs. To quantitatively assess these shape changes, cells were labeled with rhodamine phalloidin and anti-CD86 antibody, and analyzed by immunofluorescence microscopy, using serial Z-section imaging (0.2 mm spacing) and 3-D rendering. Because the expression of CD86 correlates with that of fascin1 (FIG. 1A-e), CD86^{high} DCs were chosen for morphological analysis. FIG. 4B shows representative images of wild type and KO CD86^{high} DCs stained with phalloidin. Orthogonal images in both xz and yz planes clearly showed that fascin1-deficient DCs were thinner than wild type. Statistical analyses using box plots (FIG. 4C) revealed a significant difference in thickness (p<0.0001). The median thickness of fascin1-deficient DCs (n=126) was 7.4 mm while that of wild type (n=196) was 10.7 mm. The difference in the thickness became even more prominent when DCs were centrifuged at 110×g for 4 min; fascin1-deficient DCs were greatly flattened, with a median thickness of 3.6 mm (n=55), while wild-type DCs were more resistant with a median thickness of 6.9 mm (n=45). These results suggest that DC stiffness may be impaired in fascin1-deficient DCs.

To determine how fascin1-deficiency affects cell spreading, we made area measurements of xy images on the ventral surface. As the box plot of FIG. 4D shows, fascin1-deficient DCs are 40% more spread: The median area covered by fascin1-deficient DCs was 290 mm² (n=95) whereas that of wild-type DCs was 205 mm² (n=121) with a statistical significance (p=0.0032). As wild-type DCs were 30% thicker than fascin1-deficient DCs, these measurements suggest that both types of DCs have roughly equal cell volumes.

The size and shape of protrusions varied widely, making simple measurements of the number and length of protrusions inappropriate for quantitative analyses. We thus measured circularity {(4pi)×(area)/(perimeter)²} of projected images generated from Z-section images, because more protrusions results in higher deviation from circularity (the value for a complete circle is 1). Box plot analyses (FIG. 4E) showed that fascin1-deficiency increased the median values of circularity from 0.39 (n=112) to 0.51 (n=120) with the statistical significance of p<0.0001, confirming that fascin1 KO DCs have fewer protrusions. The finding that fascin1 null DCs show reduced numbers of protrusions is consistent with previous studies demonstrating the role of fascin1 in generating membrane protrusions in other cell types (16, 26, 29, 39, 40).

### Fascin1-Deficient DCs Fail to Disassemble Podosomes Upon Maturation.

Podosomes are disassembled in mature DCs (4, 10, 11). The images at the ventral surface of fascin1 KO DCs (FIGS. 1D-h & 4B-b) showed many more podosome-like F-actin dots than in wild-type DCs, suggesting a difference in podosome dynamics. We found that this is the case. FIG. 5A shows immunofluorescence images of vinculin-labeled (red) immature and mature DCs from wild type and fascin KO mice. In each case, DCs were identified by counterstaining with CD11c (green). Immature DCs from both wild type and fascin1 KO DCs assembled podosomes to a similar extent. This result is consistent with the observation that fascin1 expression is minimal in immature DCs. As reported (4, 11), podosomes disappeared in most mature wild-type DCs. In sharp contrast, mature fascin1 KO DCs retained podosomes. We performed quantitative analyses of podosome number in CD11c-positive DCs by setting a criterion that DCs with a cluster of more than 5 podosomes (defined as vinculin-positive ring-like structures) were judged as podosome-positive. Such measurements (C) revealed that, while the percentage of wild-type DCs with podosomes decreased from 65% (n=117) to 22% (n=166) upon maturation, the percentage of podosome-positive, fascin1-deficient DCs was unchanged by maturation (61% for immature, n=158 and 59% for mature DCs, n=111).

We found that the loss of podosomes is highly correlated with the extent of fascin1 expression. As shown in our FACS analyses (FIG. 1A, d & e), mature wild type DCs can be grouped into two populations, one (about 40-50% of mature wild-type DCs) shows two orders of magnitude higher expression of fascin1 than the other. We examined the presence of podosomes in such fascin1^{high} DCs by double staining with the anti-fascin1 and anti-vinculin antibodies (FIG. 5B). We found that virtually all DCs expressing the higher level of fascin 1 (n=100) had no podosomes (pink bar, FIG. 5C), suggesting an important role for fascin1 in podosome loss. This notion is consistent with the observation that the timing of fascin1 induction (about 7 hr after LPS treatment) roughly corresponds to the time when mature DCs lose podosomes. The high correlation between high fascin1 expression and podosome loss may point to a specialized DC subset or maturation state with high fascin1 expression.

### Forced Expression of Fascin1 Results in Podosome Loss.

The above correlation has prompted us to test whether very high expression of fascin1 in mature DCs is responsible for podosome loss. To this end, we forced expression of GFP-fascin1 in fascin1-deficient DCs and counterstained them with the anti-vinculin antibody. As a control, GFP alone was transfected in a similar way. As FIG. 5D (d-f) shows, the introduction of GFP-fascin1 resulted in podosome loss in most DCs. In contrast, podosomes remained assembled in fascin1 KO DCs expressing control GFP (a-c). Measurements of podosomes in transfected cells (FIG. 5E) revealed that most (76%) of DCs expressing GFP-fascin1 (n=113) exhibited 4 or fewer podosomes while only 28% of DCs expressing control GFP (n=45) displayed fewer than 4 podosomes. These results suggest that high levels of fascin1 in mature DCs are responsible for podosome loss in mature DCs.

### Actin Bundling by Fascin1 is Critical for Podosome Loss.

We next examined whether podosome loss depends on the actin-bundling activity of fascin1. Actin-bundling activity of fascin1 is largely down-regulated by phosphorylation at Ser39 (41, 42), because phosphorylation at Ser39 disrupts one of the two actin binding sites of fascin1. We thus expressed unphosphorylatable (A-fascin1, replacing Ser39 with Ala) and phosphomimetic (D-fascin1, replacing Ser39 with Asp) mutants in fascin1 null DCs. As shown in FIG. 5D, g-l, A-fascin1 was much more effective than D-fascin1 at inducing podosome loss. Indeed, quantitative data (FIG. 5E) revealed that A-fascin1 (n=102) was slightly more effective in inducing podosome loss than wild-type fascin1 (W-fascin1), increasing the fraction of DCs without podosomes from 76±8% to 85±6% (p=0.006). In contrast, D-fascin (n=138) was much less effective than wild-type fascin, resulting in only 42±8% of the DCs displaying fewer than 4 podosomes (p<0.0001). These results indicate that the actin-bundling activity of fascin1 is important for podosome disassembly, and suggest that phosphorylation of fascin1 could contribute to the regulation of podosome assembly.

Fascin1 is Associated with the Actin Structure of Podosomes.

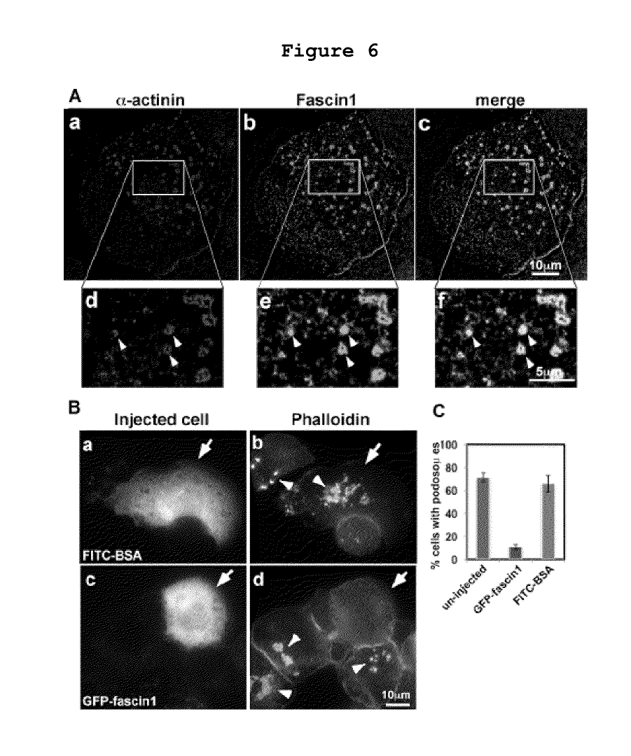

To explore how high fascin1 expression leads to podosome disassembly, we examined whether fascin1 binds to F-actin within podosomes. Because high expression of fascin1 in wild type DCs makes it difficult to determine possible localization of fascin1 at podosomes in these cells, we searched for a hematopoietic cell line that expresses a low level of fascin1 and, at the same time, has podosome structures. We found that in contrast to primary macrophages, THP-1 cells (human monocytic leukemia cells) express a low level of fascin1, yet assemble podosomes when differentiated into macrophages by addition of phorbol ester (33). Double labeling of THP-1 cells with anti-fascin1 and anti-α-actinin antibodies (FIG. 6A) clearly revealed co-localization of fascin1 (green) and α-actinin (red) at podosomes (arrowheads) with fascin1 being slightly inside the α-actinin ring structure.

The level of fascin1 in THP-1 cells is approximately 10 times lower than that of fascin1^{high} mature DCs. We thus asked whether an increase in fascin1 concentration to the level observed with DCs could result in podosome disassembly in THP-1 cells. As FIG. 6B shows, microinjection of GFP-fascin1 induced podosome disassembly (c & d) in most (89%, 50 out of 56 injected cells) THP-1 cells within 1 hr (see FIG. 6C for quantitative data). Concomitantly, fascin1-injected cells frequently became rounded and detached from the substrate if incubated for a longer time. In contrast, most (66%, 38 out of 58 injected cells) of control cells injected with FITC-labeled BSA retained podosomes (a & b of FIG. 6B), the percentage of which is statistically similar to that of un-injected cells (72%, see FIG. 6C). These results indicate that whereas fascin1 at a low concentration can bind to actin structure of the podosomes, high fascin1 expression as observed in DCs can cause podosome disassembly.

### Discussion

We have demonstrated that fascin1 plays a critical role in the alterations in motility, morphology and adhesion associated with DC maturation. Fascin1 null DCs, when fully matured, are more spread, show fewer and less dynamic membrane protrusions, and retain podosomes. Importantly, fascin1 null DCs show reduced directed migration both in vitro and in vivo.

### Fascin1 is Critical for Dynamic Dorsal Ruffling.

How does fascin1 enhance the dynamics of membrane protrusions? We found that fascin1 is co-localized with F-actin at the cortex of veil-like protrusions (FIG. 1D). Judging from its actin bundling and cross-linking activity, fascin1 is likely to form a meshwork of actin filaments at the cortex, which would give the cell cortical rigidity. Recent studies have shown that the actin cross-linking activity of fascin1 is extremely dynamic (29, 43, 44), and suggested that this dynamic cross-linking is required for vigorous movements of filopodia while at the same time maintaining sufficient rigidity for filopodial protrusions (43). A similar mechanism is likely to work for veil-like protrusions of mature DCs. As the DC cortex protrudes and retracts, fascin1 would be able to quickly reorganize the actin meshwork, providing both the rigidity and the flexibility needed to support dynamic membrane protrusions. Such dynamics are likely to be critical for DC migration through tissues and extracellular matrix to reach the lymph nodes. In keeping with this idea, we found that Langerhans cells from fascin1 KO mice show reduced emigration into draining lymph nodes (FIGS. 3B-D).

### High Fascin1 Expression is Likely to Cause the Disassembly of Podosomes in Mature DCs.

We found that fascin1 expression is closely correlated with the loss of podosomes in mature DCs. Importantly, forced expression of fascin1 in fascin1 null DCs resulted in podosome disassembly (FIG. 5). Two recent studies, however, have shown that fascin1 appears to favor assembly of podosomes, as well as invadopodia, structures closely related to podosomes (45, 46). In PDGF-treated smooth muscle cells, fascin1 depletion has been reported to suppress podosome assembly (45). Likewise, fascin1 has been shown to stabilize F-actin in invadopodia in melanoma cells (46). We speculate that these apparently contradictory functions of fascin1 may be explained by the difference in fascin1 expression levels between mature DCs and other cell types. Mature DCs express fascin1 one order in magnitude higher than do other cells. In support of this notion, we demonstrated that podosome assembly could be controlled by altering fascin1 levels in THP-1 cells: While endogenous fascin1 is present at a low level in THP-1 cells and localized to podosomes, microinjection of a large amount of fascin1 caused disassembly of podosomes (FIGS. 6B & C).

An important question is how high levels of fascin1 could cause podosome disassembly in mature DCs. Fascin1 at a low level binds to actin structure of podosomes (FIG. 6A), indicating that fascin1 and other actin binding proteins can simultaneously bind to actin filaments. However, very high levels of fascin1 would saturate actin filaments (fascin1 can bind to actin at a molar ratio of one fascin1 to four actin molecules (47)), which would compete with other proteins for actin binding (48). Such competition would result in dissociation of an actin binding protein(s) that is critical for the organization of podosomes, leading to disassembly of podosomes. Indeed, Park et al., have shown that fascin1 de-branches Arp2/3 complex-mediated branched filaments, transforming the dendritic filament assembly into actin bundles in vitro (49). Because Arp2/3 complex is an essential component of podosomes (50-53), de-branching of Arp2/3-mediated dendritic filaments could block de novo synthesis and/or maintenance of podosomes. This idea is consistent with the result that A-fascin1 is much more effective in podosome disassembly than is D-fascin1 because D-fascin1 shows much weaker actin bundling activity (FIG. 3).

### Loss of Podosomes May be Critical for Migration of Mature DCs.

Podosomes appear to profoundly alter migration patterns of DCs, at least in vitro. It has been reported that mature DCs without podosomes display “high-speed migration” with low adhesion to the substrate when compared with immature DCs with podosomes (10). Such “high speed migration” with reduced adhesion would be advantageous for mature DCs to travel to a lymph node as quickly as possible for efficient presentation of antigens to naïve T-cells. On the other hand, immature DCs need to move around the peripheral tissues in order to constantly sample foreign and host antigens. Such movement may require an adhesion structure like podosomes for attachment to the extracellular matrix (10, 13, 14, 54, 55). It is worth noting that other primary hematopoietic cells like macrophages have prominent podosomes, while no fascin1 expression was detected. These cells may need podosomes as adhesion structures so that they can move around the peripheral tissues as a sentinel against external pathogens.

The loss of podosomes might also be critical for the assembly of an immunological synapse. Geyeregger et al. have shown that agonists of Liver X receptors (LXRs) blocked fascin1 expression in human DCs and, at the same time, inhibited the assembly of the immunological synapse (56). Importantly, overexpression of fascin1 in LXR agonist-treated DCs restored immunological synapse assembly (56). We have found that LXR agonists block podosome disassembly in mature, wild type DCs (SY, unpublished result), again supporting our notion that the loss of fascin is highly correlated with sustained podosome assembly. Perhaps, the disassembly of podosomes may facilitate the assembly of an immunological synapse because these two structures share molecular constituents (57).

### Conclusion

We have demonstrated that fascin1 plays a critical role in chemotactic migration of DCs. Manipulation of fascin1 expression may thus be effective in enhancing DC-based immune therapy. For example, while tumor antigen-loaded DCs have been used as cancer vaccines, only a tiny fraction (1%) of DCs subcutaneously injected are able to migrate into lymph nodes of cancer patients (58). Accordingly, the efficiency of DC migration can be increased by either selecting DCs with high fascin1 expression and/or by exogenously increasing fascin1 expression.

### REFERENCES FOR EXAMPLE I

1. Benvenuti, F., S. Hugues, M. Walmsley, S. Ruf, L. Fetler, M. Popoff, V. L. Tybulewicz, and S. Amigorena. 2004. Requirement of Rac1 and Rac2 expression by mature dendritic cells for T cell priming. Science 305:1150-1153.
2. Calle, Y., H. C. Chou, A. J. Thrasher, and G. E. Jones. 2004. Wiskott-Aldrich syndrome protein and the cytoskeletal dynamics of dendritic cells. J Pathol 204:460-469.
3. Binks, M., G. E. Jones, P. M. Brickell, C. Kinnon, D. R. Katz, and A. J. Thrasher. 1998. Intrinsic dendritic cell abnormalities in Wiskott-Aldrich syndrome. Eur J Immunol 28:3259-3267.
4. West, M. A., R. P. Wallin, S. P. Matthews, H. G. Svensson, R. Zaru, H. G. Ljunggren, A. R. Prescott, and C. Watts. 2004. Enhanced dendritic cell antigen capture via toll-like receptor-induced actin remodeling. Science 305:1153-1157.
5. Al-Alwan, M. M., G. Rowden, T. D. Lee, and K. A. West. 2001. The dendritic cell cytoskeleton is critical for the formation of the immunological synapse. J Immunol 166:1452-1456.
6. Sims, T. N., T. J. Soos, H. S. Xenias, B. Dubin-Thaler, J. M. Hofman, J. C. Waite, T. O. Cameron, V. K. Thomas, R. Varma, C. H. Wiggins, M. P. Sheetz, D. R. Littman, and M. L. Dustin. 2007. Opposing effects of PKCtheta and WASp on symmetry breaking and relocation of the immunological synapse. Cell 129:773-785.
7. Nobes, C. D., and A. Hall. 1995. Rho, rac, and cdc42 GTPases regulate the assembly of multimolecular focal complexes associated with actin stress fibers, lamellipodia, and filopodia. Cell 81:53-62.
8. West, M. A., A. R. Prescott, E. L. Eskelinen, A. J. Ridley, and C. Watts. 2000. Rac is required for constitutive macropinocytosis by dendritic cells but does not control its downregulation. Curr Biol 10:839-848.
9. Garrett, W. S., L. M. Chen, R. Kroschewski, M. Ebersold, S. Turley, S. Trombetta, J. E. Galan, and I. Mellman. 2000. Developmental control of endocytosis in dendritic cells by Cdc42. Cell 102:325-334.
10. van Helden, S. F., D. J. Krooshoop, K. C. Broers, R. A. Raymakers, C. G. Figdor, and F. N. van Leeuwen. 2006. A critical role for prostaglandin E2 in podosome dissolution and induction of high-speed migration during dendritic cell maturation. J Immunol 177:1567-1574.
11. Burns, S., S. J. Hardy, J. Buddle, K. L. Yong, G. E. Jones, and A. J. Thrasher. 2004. Maturation of DC is associated with changes in motile characteristics and adherence. Cell Motil Cytoskeleton 57:118-132.
12. West, M. A., A. R. Prescott, K. M. Chan, Z. Zhou, S. Rose-John, J. Scheller, and C. Watts. 2008. TLR ligand-induced podosome disassembly in dendritic cells is ADAM17 dependent. J Cell Biol 182:993-1005.
13. Buccione, R., J. D. Orth, and M. A. McNiven. 2004. Foot and mouth: podosomes, invadopodia and circular dorsal ruffles. Nat Rev Mol Cell Biol 5:647-657.
14. Linder, S., and M. Aepfelbacher. 2003. Podosomes: adhesion hot-spots of invasive cells. Trends Cell Biol 13:376-385.
15. van Helden, S. F., M. M. Oud, B. Joosten, N. Peterse, C. G. Figdor, and F. N. van Leeuwen. 2008. PGE2-mediated podosome loss in dendritic cells is dependent on actomyosin contraction downstream of the RhoA-Rho-kinase axis. J Cell Sci 121:1096-1106.
16. Adams, J. C. 2004. Roles of fascin in cell adhesion and motility. Curr Opin Cell Biol 16:590-596.
17. Mosialos, G., M. Birkenbach, S. Ayehunie, F. Matsumura, G. S. Pinkus, E. Kieff, and E. Langhoff. 1996. Circulating human dendritic cells differentially express high levels of a 55-kd actin-bundling protein. Am J Pathol 148:593-600.
18. Said, J. W., J. L. Pinkus, I. P. Shintaku, S. deVos, F. Matsumura, S. Yamashiro, and G. S. Pinkus. 1998. Alterations in fascin-expressing germinal center dendritic cells in neoplastic follicles of B-cell lymphomas. Mod Pathol 11:1-5.
19. Ross, R., H. Jonuleit, M. Bros, X. L. Ross, S. Yamashiro, F. Matsumura, A. H. Enk, J. Knop, and A. B. Reske-Kunz. 2000. Expression of the actin-bundling protein fascin in cultured human dendritic cells correlates with dendritic morphology and cell differentiation. J Invest Dermatol 115:658-663.
20. Ross, R., X. L. Ross, J. Schwing, T. Langin, and A. B. Reske-Kunz. 1998. The actin-bundling protein fascin is involved in the formation of dendritic processes in maturing epidermal Langerhans cells. J Immunol 160:3776-3782.
21. Otto, J. J., R. E. Kane, and J. Bryan. 1979. Formation of filopodia in coelomocytes: localization of fascin, a 58,000 dalton actin cross-linking protein. Cell 17:285-293.
22. Adams, J. C. 1995. Formation of stable microspikes containing actin and the 55 kDa actin bundling protein, fascin, is a consequence of cell adhesion to thrombospondin-1: implications for the anti-adhesive activities of thrombospondin-1. J Cell Sci 108:1977-1990.
23. Adams, J. C., and M. A. Schwartz. 2000. Stimulation of fascin spikes by thrombospondin-1 is mediated by the GTPases Rac and Cdc42 [see comments]. J Cell Biol 150:807-822.
24. Mosialos, G., S. Yamashiro, R. W. Baughman, P. Matsudaira, L. Vara, F. Matsumura, E. Kieff, and M. Birkenbach. 1994. Epstein-Barr virus infection induces expression in B lymphocytes of a novel gene encoding an evolutionarily conserved 55-kilodalton actin-bundling protein. J Virol 68:7320-7328.
25. Yamashiro-Matsumura, S., and F. Matsumura. 1986. Intracellular localization of the 55-kD actin-bundling protein in cultured cells: spatial relationships with actin, alpha-actinin, tropomyosin, and fimbrin. J Cell Biol 103:631-640.
26. Yamashiro, S., Y. Yamakita, S. Ono, and F. Matsumura. 1998. Fascin, an actin-bundling protein, induces membrane protrusions and increases cell motility of epithelial cells. Mol. Biol. Cell 9:993-1006.
27. Hashimoto, Y., M. Skacel, and J. C. Adams. 2005. Roles of fascin in human carcinoma motility and signaling: prospects for a novel biomarker? Int J Biochem Cell Biol 37:1787-1804.
28. Vignjevic, D., M. Schoumacher, N. Gavert, K. P. Janssen, G. Jih, M. Lae, D. Louvard, A. Ben-Ze'ev, and S. Robine. 2007. Fascin, a novel target of beta-catenin-TCF signaling, is expressed at the invasive front of human colon cancer. Cancer Res 67:6844-6853.
29. Vignjevic, D., S.- i. Kojima, Y. Aratyn, O. Danciu, T. Svitkina, and G. G. Borisy. 2006. Role of fascin in filopodial protrusion. J. Cell Biol. 174:863-875.
30. Al-Alwan, M. M., G. Rowden, T. D. Lee, and K. A. West. 2001. Fascin is involved in the antigen presentation activity of mature dendritic cells. J Immunol 166:338-345.
31. Yamakita, Y., F. Matsumura, and S. Yamashiro. 2009. Fascin1 is dispensable for mouse development but is favorable for neonatal survival. Cell Motil Cytoskeleton 66:524-534.
32. Inaba, K., M. Inaba, N. Romani, H. Aya, M. Deguchi, S. Ikehara, S. Muramatsu, and R. M. Steinman. 1992. Generation of large numbers of dendritic cells from mouse bone marrow cultures supplemented with granulocyte/macrophage colony-stimulating factor. J Exp Med 176:1693-1702.
33. Tsuchiya, S., Y. Kobayashi, Y. Goto, H. Okumura, S. Nakae, T. Konno, and K. Tada. 1982. Induction of maturation in cultured human monocytic leukemia cells by a phorbol diester. Cancer Res 42:1530-1536.
34. Price, A. A., M. Cumberbatch, I. Kimber, and A. Ager. 1997. Alpha 6 integrins are required for Langerhans cell migration from the epidermis. J Exp Med 186:1725-1735.
35. Wang, B., L. Zhuang, H. Fujisawa, G. A. Shinder, C. Feliciani, G. M. Shivji, H. Suzuki, P. Amerio, P. Toto, and D. N. Sauder. 1999. Enhanced epidermal Langerhans cell migration in IL-10 knockout mice. J Immunol 162:277-283.
36. Stoitzner, P., C. H. Tripp, P. Douillard, S. Saeland, and N. Romani. 2005. Migratory Langerhans cells in mouse lymph nodes in steady state and inflammation. J Invest Dermatol 125:116-125.
37. Duh, F. M., F. Latif, Y. Weng, L. Geil, W. Modi, T. Stackhouse, F. Matsumura, D. R. Duan, W. M. Linehan, M. I. Lerman, and et al. 1994. cDNA cloning and expression of the human homolog of the sea urchin fascin and Drosophila singed genes which encodes an actin-bundling protein. DNA Cell Biol 13:821-827.
38. Pinkus, G. S., J. L. Pinkus, E. Langhoff, F. Matsumura, S. Yamashiro, G. Mosialos, and J. W. Said. 1997. Fascin, a sensitive new marker for Reed-Sternberg cells of Hodgkin's Disease. Evidence for a dendritic or B cell derivation? Am J Pathol 150:543-562.
39. Hashimoto, Y., M. Parsons, and J. C. Adams. 2007. Dual actin-bundling and protein kinase C-binding activities of fascin regulate carcinoma cell migration downstream of Rac and contribute to metastasis. Mol. Biol. Cell 18:4591-4602.
40. Otto, J. J., R. E. Kane, and J. Bryan. 1980. Redistribution of actin and fascin in sea urchin eggs after fertilization. Cell Motil 1:31-40.
41. Ono, S., Y. Yamakita, S. Yamashiro, P. T. Matsudaira, J. R. Gnarra, T. Obinata, and F. Matsumura. 1997. Identification of an actin binding region and a protein kinase C phosphorylation site on human fascin. J Biol Chem 272:2527-2533.
42. Yamakita, Y., S. Ono, F. Matsumura, and S. Yamashiro. 1996. Phosphorylation of human fascin inhibits its actin binding and bundling activities. J Biol Chem 271:12632-12638.
43. Aratyn, Y. S., T. E. Schaus, E. W. Taylor, and G. G. Borisy. 2007. Intrinsic Dynamic Behavior of Fascin in Filopodia. Mol. Biol. Cell % R 10.1091/mbc.E07-04-0346:E07-04-0346.
44. Nakagawa, H., A. G. Terasaki, H. Suzuki, K. Ohashi, and S. Miyamoto. 2006. Short-term retention of actin filament binding proteins on lamellipodial actin bundles. FEBS Lett 580:3223-3228.
45. Quintavalle, M., L. Elia, G. Condorelli, and S. A. Courtneidge. 2010. MicroRNA control of podosome formation in vascular smooth muscle cells in vivo and in vitro. J Cell Biol 189:13-22.
46. Li, A., J. C. Dawson, M. Forero-Vargas, H. J. Spence, X. Yu, I. Konig, K. Anderson, and L. M. Machesky. 2010. The actin-bundling protein fascin stabilizes actin in invadopodia and potentiates protrusive invasion. Curr Biol 20:339-345.
47. Yamashiro-Matsumura, S., and F. Matsumura. 1985. Purification and characterization of an F-actin-bundling 55-kilodalton protein from HeLa cells. J Biol Chem 260:5087-5097.
48. Ishikawa, R., S. Yamashiro, K. Kohama, and F. Matsumura. 1998. Regulation of actin binding and actin bundling activities of fascin by caldesmon coupled with tropomyosin. J Biol Chem 273:26991-26997.
49. Park, S. J., S. Suetsugu, H. Sagara, and T. Takenawa. 2007. HSP90 cross-links branched actin filaments induced by N-WASP and the Arp2/3 complex. Genes Cells 12:611-622.
50. Morita, T., T. Mayanagi, T. Yoshio, and K. Sobue. 2007. Changes in the balance between caldesmon regulated by p21-activated kinases and the Arp2/3 complex govern podosome formation. J Biol Chem 282:8454-8463.
51. Kaverina, I., T. E. Stradal, and M. Gimona. 2003. Podosome formation in cultured A7r5 vascular smooth muscle cells requires Arp2/3-dependent de-novo actin polymerization at discrete microdomains. J Cell Sci 116:4915-4924.
52. Tsuboi, S. 2007. Requirement for a complex of Wiskott-Aldrich syndrome protein (WASP) with WASP interacting protein in podosome formation in macrophages. J Immunol 178:2987-2995.
53. Olivier, A., L. Jeanson-Leh, G. Bouma, D. Compagno, J. Blondeau, K. Seye, S. Charrier, S. Burns, A. J. Thrasher, O. Danos, W. Vainchenker, and A. Galy. 2006. A partial down-regulation of WASP is sufficient to inhibit podosome formation in dendritic cells. Mol Ther 13:729-737.
54. Calle, Y., S. Burns, A. J. Thrasher, and G. E. Jones. 2006. The leukocyte podosome. Eur Cell Biol 85:151-157.
55. Linder, S. 2007. The matrix corroded: podosomes and invadopodia in extracellular matrix degradation. Trends Cell Biol 17:107-117.
56. Geyeregger, R., M. Zeyda, W. Bauer, E. Kriehuber, M. D. Saemann, G. J. Zlabinger, D. Maurer, and T. M. Stulnig. 2007. Liver X receptors regulate dendritic cell phenotype and function through blocked induction of the actin-bundling protein fascin. Blood 109:4288-4295.
57. Wernimont, S. A., C. L. Cortesio, W. T. Simonson, and A. Huttenlocher. 2008. Adhesions ring: a structural comparison between podosomes and the immune synapse. Eur J Cell Biol 87:507-515.
58. De Vries, I. J., D. J. Krooshoop, N. M. Scharenborg, W. J. Lesterhuis, J. H. Diepstra, G. N. Van Muijen, S. P. Strijk, T. J. Ruers, O. C. Boerman, W. J. Oyen, G. J. Adema, C. J. Punt, and C. G. Figdor. 2003. Effective migration of antigen-pulsed dendritic cells to lymph nodes in melanoma patients is determined by their maturation state. Cancer Res 63:12-17.

### Example II

### Fascin1 Confers Resistance to Listeria monocytogenes (Lm) in DCs

As set forth in Example I, Fascin1 is essential for dendrite formation, antigen presentation and immunological synapse formation of DCs. While fascin1 is generally thought to be involved in the assembly of filopodia, an increasing body of evidence indicates that fascin1 plays a specific and unique role(s) in DC biology by inducing massive alterations in actin organization. We and others have found that (1) fascin1 expression is specifically induced in DCs upon maturation; and (2) fascin1 depletion by anti-sense treatment results in the inhibition of both dendrite formation and T-cell activation in bone marrow-derived DCs. Consistent with these observations, fascin1 protein is highly enriched at the immunological synapse (IS), and is necessary for the assembly of the IS [10][15].

We have also identified a critical role of DCs in immunity against Listeria monocytogenes (Lm). Lm is a pathogen that can cause serious infections in immunocompromised individuals and pregnant women. Lm enters DCs via phagocytosis. Upon entry, the bacterium lyses the primary vacuole with listeriolysin O (LLO), a pore-forming cytolysin, and escapes into the cytoplasm for proliferation. This pathogen also polymerizes actin for rapid intracellular movement, and transmigrates directly into adjacent cells to promote new infections.

DCs have been demonstrated to be essential for eradication of Lm in mice: DCs, but not macrophages, play crucial roles in priming naive cytotoxic T-lymphocytes that are specific to Lm antigens. In vitro studies of Lm infection of DCs revealed that cytoplasmic entry of Lm enhances maturation of DCs, as well as T-cell differentiation. Interestingly, a subpopulation of DCs has been shown to be less susceptible to Lm than macrophages. This resistance appears to be due to the higher ability of a subpopulation of DCs to kill the bacteria than macrophages.

Taken together, these results indicate the critical role of the DCs' resistance in protection against Lm infection: Lm infection of immature DCs induces DC maturation and, at the same time, the subpopulation of DCs survives the Lm infection by killing the bacteria, allowing such mature DCs to present bacterial antigens to cytotoxic T-cells, which ultimately eradicates Lm. While the mechanism is unclear, it has been suggested that a subpopulation of DCs is able to encapsulate the bacterium within vacuoles, thereby prohibiting release of bacteria into the cytoplasm and leading to bacterial clearance.

### Key Roles of Fascin1 in the Maturation and Motility of Mature Dendritic Cells

Our characterization of fascin1 KO DCs revealed crucial roles of fascin1 in the massive cytoskeletal reorganization induced by DC maturation. We found that fascin1 KO DCs are thinner and more spread with much less dorsal ruffling than wild type DCs (See Example 1). Furthermore, fascin1 KO DCs fail to disassemble podosomes (a specialized structure for adhesion to a substrate) and show much reduced migration in vitro, as well as in vivo (examined by migration of Langerhans cells, skin DCs, into draining lymph nodes). Importantly, these defects are rescued by reintroduction of GFP-fascin1, indicating that fascin1 is responsible for the maturation-induced, alterations in cytoskeleton and motility. FACS analyses revealed that both wild type and fascin1 KO DCs express similar levels of DC maturation markers (MHC-II, CD86), indicating that maturation-induced gene expression occurs normally. Importantly a subpopulation (about 30-40%) of wild type DCs show very high fascin1 expression upon maturation.

### Fascin1 Confers Resistance to Lm.

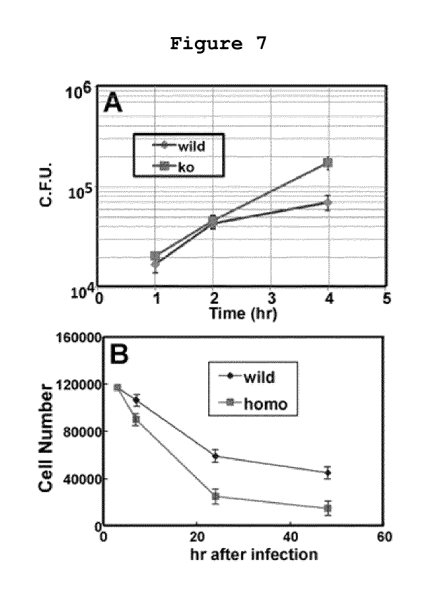

We hypothesized that the massive reorganization of the cytoskeleton caused by fascin1 may affect actin-mediated host defense processes. To test this hypothesis, we infected immature DCs with Lm. Wild-type, as well as fascin1 null DCs, were infected for 30 minutes with Lm with a multiplicity of infection (moi) of 0.5, and further infection was blocked by gentamicin addition. Intracellular bacterial numbers were measured at 1 hr, 2 hr and 4 hr post infection to determine replication rates. As FIG. 7A shows, Lm replicated faster in fascin1 KO DCs than in wild type DCs. During the initial 2 hr, Lm grew in a similar rate in both wild type and fascin1 KO DCs. However, Lm growth became much slower at 4 hr in wild type DCs, whereas Lm grew consistently in KO DCs. The doubling times (averaged between 1 and 4 hr) were estimated to be 50 min in fascin1 KO DCs and 90 min in wild type DCs. This 90 min doubling time in wild type DCs, as well as the growth inhibition at 4 hr post infection, is consistent to the previous report (70 min), verifying our analyses of Lm replication. The susceptibility to Lm infection in fascin1 deficient DCs is further confirmed by tracking viability of DCs for over 24 hr: As FIG. 7B shows, while fascin1 null DCs were killed by Lm infection, about one-third of wild type DCs survived.

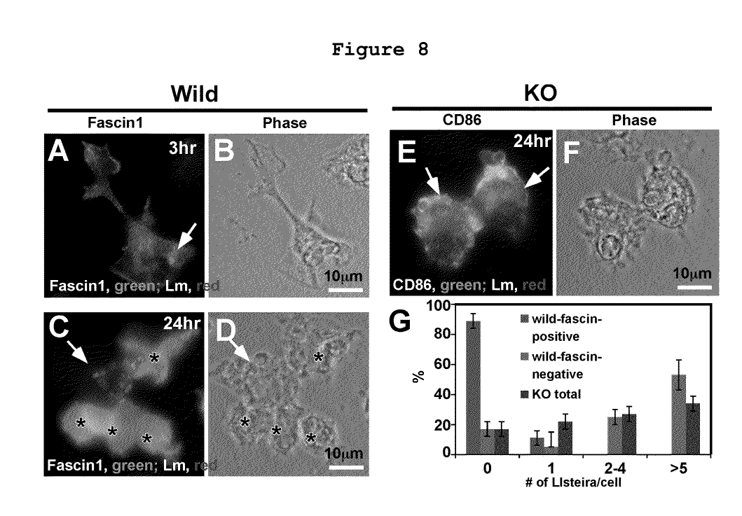

To further examine the correlation between fascin1 expression and the resistance to Lm, wild type DCs were infected with a high moi of 5 and labeled with the anti-fascin1 and anti-Lm antibodies at 3 hr and 24 hr post infection (FIG. 8 A-D). Consistent with previous reports [13, 14], Lm infection induced DC maturation, as judged from the much-increased expression of MHC-II and CD86 at 3 h post infection (data not shown). Fascin1 was induced at the same time (FIG. 8A). Importantly, this timing of fascin1 induction roughly corresponds to the time when Lm growth was inhibited in wild type DCs (see FIG. 7A), supporting our hypothesis. At 3 hr post infection (FIGS. 8A & B), we found that wild type DCs with fascin1 expression (labeled green) were infected with one or two Lm (labeled red). At 24 hr post infection (C & D), however, DCs with high expression of fascin1 (indicated by asterisks) cleared Lm while adjacent fascin1-negative DCs (indicated by arrow) were heavily infected.

In contrast, fascin1 KO DCs were heavily infected with Lm at both 3 hr (data not shown) and 24 hr post infection (E & F, note that the green in E indicates CD86, a maturation marker, not fascin1). Quantitative analyses (G) confirmed that, at 24 hr post infection, 90% of DCs with high fascin1 expression (red bars) cleared Lm, whereas more than 80% of fascin1-negative, wild-type DCs (green bars) were heavily infected. Consistently, fascin1 null DCs (blue bars) were heavily infected with Lm, similar to that of fascin1-negative wild type DCs. Taken together, these results suggest that fascin1 is a part of the molecular system that confers the resistance to Lm infection in DCs, and that wild type DCs appear to constitute two populations: one is Lm-free DCs with high fascin1 expression and the other is heavily infected DCs with low or unrecognizable fascin1 expression. We propose that the fascin1-expressing DCs are likely to correspond to the previously reported subpopulation (about one-third) of wild type DCs that are able to clear Lm infection [3].

### Fascin1 Facilitates Vacuolar Acidification.

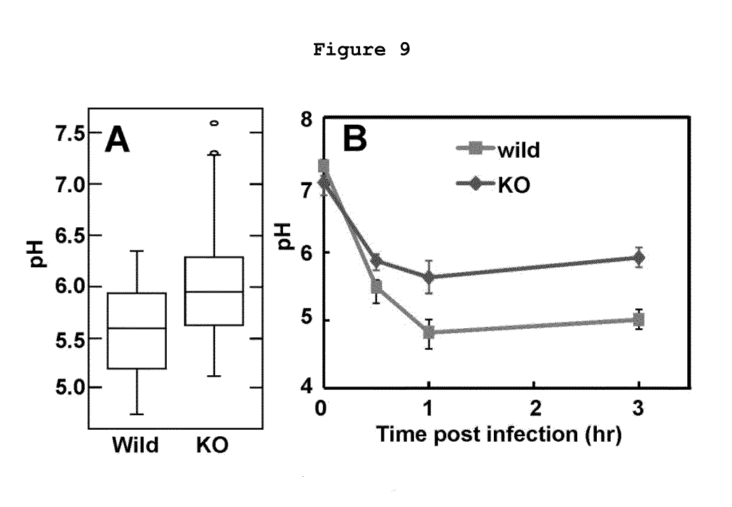

One possibility for the higher killing activity is that fascin1 accelerates phagolysosomal fusion. Because the fusion depends on phagosomal acidification [16], we measured vacuolar pH by two independent methods. We found that wild type DCs show lower vacuolar pH than fascin1 KO DCs (FIG. 9).

The first method was to use fluorescently labeled Lm as a pH indicator (Lm double labeled Lm with a pH-sensitive (CMFDA) and pH-insensitive (Rhodamine) dyes). In addition, an Lm mutant (DP L-2319, Dhly DplcA DplcB, that cannot escape from phagosomes; provided by D. Portnoy) was used to ensure measurements of pH in phagosomes. As FIG. 9A shows, the phagosomal pH measured at 1.5 hr post infection was lower in wild type DCs than in fascin1 KO DCs: the pH values were 5.6 (n=44) for wild type DCs and 6.0 (n=87) for fascin1 KO DCs (p<0.0001). The pH value (pH 5.6) of wild type DCs is consistent with that recently reported by Westcott et al [17], supporting that fascin1 is critical for phagosomal acidification. As the second approach, we used a LysoSensor yellow/blue dye (DN-160, Molecular Probes). This dye is incorporated into acidic organelles (including phagosomes, autophagosomes and lysosomes), and ratio imaging of 525 nm/470 nm gives pH values. As FIG. 9B shows, Lm infection induced acidification of the vacuolar pH in wild type DCs to a greater extent than it did in fascin1 KO DCs. Prior to infection, the vacuolar pH was neutral to alkaline (7.0-7.2), which is consistent with the previous report that the phagosomal pH in immature DCs is alkaline [18]. At 1-3 hr post infection, however, the vacuolar pH of wild type DCs became acidic to the range between 4.6 and 5.0. In contrast, the vacuolar pH of fascin1 KO DCs was less lowered to the range between 5.4 and 5.8 during the same time window. These differences in the pH values between wild type and fascin1 KO DCs are statistically significant (p<0.0001).

The actin cytoskeleton has been reported to play several roles in host defense systems. First, actin is involved in lysosomal fusion of late endosomes, which is likely to be critical for killing of phagosome-encapsulated bacteria by lysosomal enzymes [19, 20]. Second, actin has been reported to be involved in autophagy-mediated killing of cytosolic bacteria [21, 22]. While autophagy is known to be an intracellular process for encapsulation and degradation of damaged organelles or proteins, it also functions as an antimicrobial system to engulf and clear cytosolic bacteria [23]. Third, the actin cytoskeleton, via ezrin-radixin-moesin-binding phosphoprotein 50 (EBP50), recruits NOS2 (also called iNOS, an inducible NO synthase) to phagosomes, thereby allowing the localized concentration of bactericidal reactive nitrogen intermediates (RNI) [24]. It is also known that bacterial pathogens manipulate these actin-mediated host-defense mechanisms for survival and proliferation [25-29][30]. These studies underscore the key roles of the actin cytoskeleton in host defense systems.

We found that fascin1 is essential for DC's resistance to Lm infection. Because fascin1 is specifically induced in a subpopulation of DCs upon Lm infection (note that fascin1 is not expressed in other blood cells including macrophages or neutrophils), fascin1 appears to play a key role in the DC-specific, actin-mediated host defense systems.

The increased vacuolar acidification in wild type DCs (FIG. 9) suggests two possible (though not mutually exclusive) functions of fascin1: One is acceleration of phagolysosomal fusion and the other is enhanced recruitment of Vacuolar-ATPase (V-ATPase) to phagosomes. V-ATPase binds to the actin cytoskeleton. Though the mechanism is not clear, actin binding is suggested to affect its localization to phagosomes and lysosomes [37-40]. Fascin1, by bundling actin filaments, could enhance targeting of V-ATPase to phagosomes, lowering their pH, thereby facilitating their maturation and lysosomal fusion.

Several different approaches can be employed to assess the role that fascin1 plays in acceleration of phagolysosomal fusion. These include, without limitation, live cell imaging, immunofluorescence microscopy and in vitro fusion assays with detectably labeled reagents. To examine effects of fascin1 on the recruitment of V-ATPase to phagosomes, phagosomes will be separated from Lm-infected DCs by density gradient centrifugation as described [42-46]. The association of V-ATPase with phagosomes will then be examined by Western blotting to see whether phagosome markers (such as Rab5 and Rab7) are co-eluted with V-ATPase (both V0 and V1 sectors) markers. More association of V1 with phagosomes in wild type DCs would indicate a role for fascin1 in the targeting V-ATPase to phagosomes, as well as its activation. This type of phagosome analysis is feasible as we can prepare 2.5×10⁷ DCs from 5 mice.

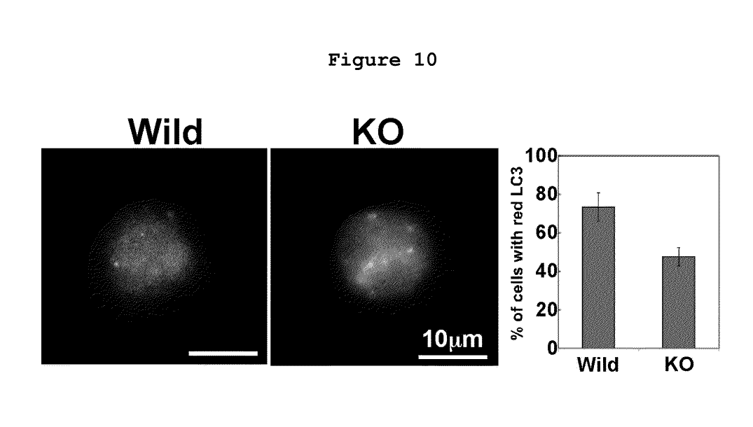

DCs must control the proliferation of Lm that have escaped from phagosomes. One such system is via autophagy, the process of which is also controlled by the actin cytoskeleton [21, 22, 48]. Preliminary experiments revealed that wild type DCs have a higher autophagy flux (fusion of autophagosomes with lysosomes) than do fascin1 KO DCs when both types of DCs are activated by LPS (FIG. 10). Autophagy flux was examined by a color change of tfLC3 (LC3 tandemly tagged with GFP and RFP) from yellow to red because autophagolysosomal fusion changes the color from yellow to red [49]. Wild type DCs showed more LC3 puncta with red color than do fascin1 KO DCs, indicating that tfLC3 is in the acidic compartments in wild type DCs. These results suggest that fascin1 enhances autophagic flux by facilitating autophagosome-lysosome fusion. Such results are also consistent with our result of the higher vacuolar acidification in wild type DCs (FIG. 9B).

Judging from the in vitro roles of fascin1 in the resistance of DCs to Lm, as well as in DC migration to draining lymph nodes and T-cell activation, we propose that fascin1 KO mice will likely show reduced adaptive immune responses to Lm infection in vivo. Because DCs have also been implicated to play roles in the innate response [52], fascin1 may also be involved in innate immunity. To test these possibilities, we will use the Listeria mouse model as described [53-56]. As fascin1 KO mice were backcrossed onto C57/BL6 for 16 generations, the LD₅₀ of littermate wild type mice should be similar to that of the C57/BL6 strain (1×10⁵ for the Lm strain 10403S, [57]).

To examine effects of fascin1 on innate immunity, wild type and fascin1 KO mice will be injected intravenously with 0.2 or 1 LD₅₀ to examine their survival. Bacterial burdens of spleens and livers will also be determined at 48-72 hr post infection Innate protection largely depends on cytokines (including TNFa, IFNg, IL12) produced by macrophages and DCs. Thus, if we find reduced innate protection in fascin1 KO mice, we will determine cytokine production in sera and spleens. For adaptive immunity, mice will be first intravenously injected with a dose of 0.1 LD₅₀, and then challenged with 5-10 LD₅₀ of Lm at two different timings of 5-7 days and 30 days. These timings are chosen because the primary CD8⁺ T-cell responses will be the maximum at 5-7 days whereas responses of memory CD8⁺ T-cells can be examined at the later timing. In both cases, bacterial numbers in spleens and livers, as well as survival rates of challenged mice, will be determined. If fascin1 deficiency reduces the adaptive immunity, we will examine, by FACS analyses, whether the expansion of CD8⁺ IFN-g⁺ T-cells is attenuated.

To directly assess the ability of DCs to induce epitope-specific CD8⁺ T-cells in an MHC class 1-restricted manner, we will use DC vaccination [61-64]. CD8⁺ T-cells from OT-1 mice that are specific to OVA₂₅₇₋₂₆₄ will be adoptively transferred into fascin1 KO mice 2 days prior to DC vaccination. Wild type and fascin1 KO DCs will be infected in vitro with Lm-OVA (a Lm strain that expresses OVA as a model antigen) and then injected into these adaptively transferred mice. Forty days later, mice will be challenged with 1-5 LD₅₀ to see whether vaccination with fascin1 KO DCs is less protective to Lm infection than vaccination with wild type DCs. Activation of MHC class I-restricted CD8⁺ T-cells in the spleens will be analyzed by FACS analyses using OVA₂₅₇₋₂₆₄/H2 Kb tetramer.

These experiments will provide critical information regarding the in vivo roles of fascin1 in host defense mechanisms. A group of five wild type and five fascin1 KO mice will be initially used for each experimental setting. Statistical significance will be determined by student's T-tests (p values less than 0.05 are considered significant), and power analyses with the setting of the power to 0.8 will be performed to determine whether the sample number is sufficient for rejection of a null hypothesis.

Our findings that Fascin1-expressing DCs are resistant to Lm points to a completely new and unexpected role for fascin1 in innate and adaptive immune responses. Our fascin1 KO mice [32] that are in hand and are fully backcrossed in an isogenic background, provide an ideal experimental system to quickly and efficiently delineate the mechanisms by which fascin1 contributes to host defense against intracellular pathogens and contributes to innate and adaptive immunity and to identify potential therapeutic agents which impact these processes.

The elucidation of the mechanisms has important implications for practical therapy development. For example, fascin1-expressing DCs could be utilized in DC-based immunotherapy [65-67]. Because such DCs are likely to show increased migration into lymph nodes, have a higher potency to activate T-cells, and are more resistant to bacterial pathogens, they, after being pulsed with a pathogen, could function as an improved immunostimulator against infectious diseases. It should be noted that the increased migration ability of fascin1-expressing DCs is particularly advantageous because a major drawback of DC vaccination is that only a tiny fraction (1%) of DCs subcutaneously injected are known to reach to the lymph nodes [68].

Fascin1-expressing DCs may also prove to be useful for the Lm-based cancer therapy: Lm, after being engineered to minimize its toxicity and to express cancer antigens, has been tested for clinical trials because Lm can induce strong immune responses against cancer [69-71]. Because of the resistance of fascin1-expressing DCs to Lm, such DCs could be used more safely for DC vaccination, and show stronger immunostimulator activity.

### REFERENCES FOR EXAMPLE 2

1. Ross, R., et al., The actin-bundling protein fascin is involved in the formation of dendritic processes in maturing epidermal Langerhans cells. J Immunol, 1998. 160(8): p. 3776-82.
2. Mosialos, G., et al., Circulating human dendritic cells differentially express high levels of a 55-kd actin-bundling protein. Am J Pathol, 1996. 148(2): p. 593-600.
3. Westcott, M. M., et al., Differential susceptibility of bone marrow-derived dendritic cells and macrophages to productive infection with Listeria monocytogenes. Cell Microbiol, 2007. 9(6): p. 1397-411.
4. Adams, J. C., Roles of fascin in cell adhesion and motility. Curr Opin Cell Biol, 2004. 16(5): p. 590-6.
5. Said, J. W., et al., Alterations in fascin-expressing germinal center dendritic cells in neoplastic follicles of B-cell lymphomas. Mod Pathol, 1998. 11(1): p. 1-5.
6. Ross, R., et al., Expression of the actin-bundling protein fascin in cultured human dendritic cells correlates with dendritic morphology and cell differentiation. J Invest Dermatol, 2000. 115(4): p. 658-63.
7. Al-Alwan, M. M., et al., Fascin is involved in the antigen presentation activity of mature dendritic cells. J Immunol, 2001. 166(1): p. 338-45.
8. Al-Alwan, M. M., et al., Cutting edge: dendritic cell actin cytoskeletal polarization during immunological synapse formation is highly antigen-dependent. J Immunol, 2003. 171(9): p. 4479-83.
9. Rothoeft, T., et al., Structure and duration of contact between dendritic cells and T cells are controlled by T cell activation state. Eur J Immunol, 2006. 36(12): p. 3105-17.
10. Geyeregger, R., et al., Liver X receptors regulate dendritic cell phenotype and function through blocked induction of the actin-bundling protein fascin. Blood, 2007. 109(10): p. 4288-95.
11. Portnoy, D. A., V. Auerbuch, and I. J. Glomski, The cell biology of Listeria monocytogenes infection: the intersection of bacterial pathogenesis and cell-mediated immunity. Cell Biol, 2002. 158(3): p. 409-14.
12. Jung, S., et al., In vivo depletion of CD11c(+) dendritic cells abrogates priming of CD8(+) T cells by exogenous cell-associated antigens. Immunity, 2002. 17(2): p. 211-20.
13. Brzoza, K. L., A. B. Rockel, and E. M. Hiltbold, Cytoplasmic entry of Listeria monocytogenes enhances dendritic cell maturation and T cell differentiation and function. J Immunol, 2004. 173(4): p. 2641-51.
14. Paschen, A., et al., Human dendritic cells infected by Listeria monocytogenes: induction of maturation, requirements for phagolysosomal escape and antigen presentation capacity. Eur J Immunol, 2000. 30(12): p. 3447-56.
15. Yamakita, Y., et al., Fascin1 promotes cell migration of mature dendritic cells. J Immunol, 2011. 186(5): p. 2850-9.
16. Kinchen, J. M. and K. S. Ravichandran, Phagosome maturation: going through the acid test. Nat Rev Mol Cell Biol, 2008. 9(10): p. 781-95.
17. Westcott, M. M., et al., Dendritic cells inhibit the progression of Listeria monocytogenes intracellular infection by retaining bacteria in major histocompatibility complex class II-rich phagosomes and by limiting cytosolic growth. Infect Immun, 2010. 78(7): p. 2956-65.
18. Savina, A., et al., NOX2 controls phagosomal pH to regulate antigen processing during crosspresentation by dendritic cells. Cell, 2006. 126(1): p. 205-18.
19. Girao, H., M. I. Geli, and F. Z. Idrissi, Actin in the endocytic pathway: from yeast to mammals. FEBS Lett, 2008. 582(14): p. 2112-9.
20. Kjeken, R., et al., Fusion between phagosomes, early and late endosomes: a role for actin in fusion between late, but not early endocytic organelles. Mol Biol Cell, 2004. 15(1): p. 345-58.
21. Monastyrska, I., et al., Arp2 links autophagic machinery with the actin cytoskeleton. Mol Biol Cell, 2008. 19(5): p. 1962-75.
22. Lee, J. Y., et al., HDAC6 controls autophagosome maturation essential for ubiquitin-selective quality-control autophagy. Embo J, 2010.
23. Yano, T. and S. Kurata, Induction of autophagy via innate bacterial recognition. Autophagy, 2008. 4(7): p. 958-60.
24. Davis, A. S., et al., Mechanism of inducible nitric oxide synthase exclusion from mycobacterial phagosomes. PLoS Pathog, 2007. 3(12): p. e186.
25. Diacovich, L. and J. P. Gorvel, Bacterial manipulation of innate immunity to promote infection. Nat Rev Microbiol, 2010. 8(2): p. 117-28.
26. Ogawa, M., et al., Escape of intracellular Shigella from autophagy. Science, 2005. 307(5710): p. 727-31.
27. Birmingham, C. L., et al., Listeria monocytogenes evades killing by autophagy during colonization of host cells. Autophagy, 2007. 3(5): p. 442-51.
28. Birmingham, C. L., et al., Listeriolysin O allows Listeria monocytogenes replication in macrophage vacuoles. Nature, 2008. 451(7176): p. 350-4.
29. Yoshikawa, Y., et al., Listeria monocytogenes ActA-mediated escape from autophagic recognition. Nat Cell Biol, 2009. 11(10): p. 1233-40.
30. Miller, B. H., et al., Mycobacteria inhibit nitric oxide synthase recruitment to phagosomes during macrophage infection. Infect Immun, 2004. 72(5): p. 2872-8.
31. Yamashiro-Matsumura, S. and F. Matsumura, Intracellular localization of the 55-kD actin-bundling protein in cultured cells: spatial relationships with actin, alpha-actinin, tropomyosin, and fimbrin. J Cell Biol, 1986. 103(2): p. 631-40.
32. Yamakita, Y., F. Matsumura, and S. Yamashiro, Fascin1 is dispensable for mouse development but is favorable for neonatal survival. Cell Motil Cytoskeleton, 2009. 66(8): p. 524-34.
33. Yamashiro-Matsumura, S. and F. Matsumura, Purification and characterization of an F-actin-bundling 55-kilodalton protein from HeLa cells. J Biol Chem, 1985. 260(8): p. 5087-97.
34. Duh, F. M., et al., cDNA cloning and expression of the human homolog of the sea urchin fascin and Drosophila singed genes which encodes an actin-bundling protein. DNA Cell Biol, 1994. 13(8): p. 821-7.
35. Ono, S., et al., Identification of an actin binding region and a protein kinase C phosphorylation site on human fascin. J Biol Chem, 1997. 272(4): p. 2527-33.
36. Yamakita, Y., et al., Phosphorylation of human fascin inhibits its actin binding and bundling activities. J Biol Chem, 1996. 271(21): p. 12632-8.
37. Vitayska, O., H. Merzendorfer, and H. Wieczorek, The V-ATPase subunit C binds to polymeric F-actin as well as to monomeric G-actin and induces cross-linking of actin filaments. J Biol Chem, 2005. 280(2): p. 1070-6.
38. Wieczorek, H., et al., The insect plasma membrane H+ V-ATPase: intra-, inter-, and supramolecular aspects. J Bioenerg Biomembr, 2003. 35(4): p. 359-66.
39. Vitayska, O., H. Wieczorek, and H. Merzendorfer, A novel role for subunit C in mediating binding of the H+-V-ATPase to the actin cytoskeleton. J Biol Chem, 2003. 278(20): p. 18499-505.
40. Holliday, L. S., et al., The amino-terminal domain of the B subunit of vacuolar H+-ATPase contains a filamentous actin binding site. J Biol Chem, 2000. 275(41): p. 32331-7.
41. Jahraus, A., et al., In vitro fusion of phagosomes with different endocytic organelles from J774 macrophages. J Biol Chem, 1998. 273(46): p. 30379-90.
42. Grotzke, J. E., et al., The Mycobacterium tuberculosis phagosome is a HLA-I processing competent organelle. PLoS Pathog, 2009. 5(4): p. e1000374.
43. Trombetta, E. S., et al., Activation of lysosomal function during dendritic cell maturation. Science, 2003. 299(5611): p. 1400-3.
44. Katti, M. K., et al., The Delta fbpA mutant derived from Mycobacterium tuberculosis H37Rv has an enhanced susceptibility to intracellular antimicrobial oxidative mechanisms, undergoes limited phagosome maturation and activates macrophages and dendritic cells. Cell Microbiol, 2008. 10(6): p. 1286-303.
45. Ullrich, H. J., W. L. Beatty, and D. G. Russell, Interaction of Mycobacterium avium-containing phagosomes with the antigen presentation pathway. J Immunol, 2000. 165(11): p. 6073-80.
46. Li, Q., et al., Analysis of phagosomal proteomes: From latex-bead to bacterial phagosomes. Proteomics, 2010: p. n/a-n/a.
47. Yamashiro, S., et al., Myosin phosphatase-targeting subunit 1 regulates mitosis by antagonizing polo-like kinase 1. Dev Cell, 2008. 14(5): p. 787-97.
48. Monastyrska, I., et al., Multiple roles of the cytoskeleton in autophagy. Biol Rev Camb Philos Soc, 2009. 84(3): p. 431-48.
49. Kimura, S., et al., Monitoring autophagy in mammalian cultured cells through the dynamics of LC3. Methods Enzymol, 2009. 452: p. 1-12.
50. Berche, P., J. L. Gaillard, and P. J. Sansonetti, Intracellular growth of Listeria monocytogenes as a prerequisite for in vivo induction of T cell-mediated immunity. J Immunol, 1987. 138(7): p. 2266-71.
51. Perrin, A. J., et al., Recognition of bacteria in the cytosol of Mammalian cells by the ubiquitin system. Curr Biol, 2004. 14(9): p. 806-11.
52. Steinman, R. M. and H. Hemmi, Dendritic cells: translating innate to adaptive immunity. Curr Top Microbiol Immunol, 2006. 311: p. 17-58.
53. Zenewicz, L. A. and H. Shen, Innate and adaptive immune responses to Listeria monocytogenes: a short overview. Microbes Infect, 2007. 9(10): p. 1208-15.
54. Pamer, E. G., Immune responses to Listeria monocytogenes. Nat Rev Immunol, 2004. 4(10): p. 812-23.
55. Geginat, G. and S. Grauling-Halama, Innate Immunity, in Handbook of Listeria Monocytogenes. 2008, CRC Press. p. 397-426.
56. Mitsuyama, M., Adaptive Immunity, in Handbook of Listeria Monocytogenes. 2008, CRC Press. p. 427-448.
57. Crimmins, G. T., et al., Listeria monocytogenes 6-Phosphogluconolactonase mutants induce increased activation of a host cytosolic surveillance pathway. Infect Immun, 2009. 77(7): p. 3014-22.
58. Rothe, J., et al., Mice lacking the tumour necrosis factor receptor 1 are resistant to TNF-mediated toxicity but highly susceptible to infection by Listeria monocytogenes. Nature, 1993. 364(6440): p. 798-802.
59. Tam, M. A. and M. J. Wick, Differential expansion, activation and effector functions of conventional and plasmacytoid dendritic cells in mouse tissues transiently infected with Listeria monocytogenes. Cell Microbiol, 2006. 8(7): p. 1172-87.
60. Tripp, C. S., S. F. Wolf, and E. R. Unanue, Interleukin 12 and tumor necrosis factor alpha are costimulators of interferon gamma production by natural killer cells in severe combined immunodeficiency mice with listeriosis, and interleukin 10 is a physiologic antagonist. Proc Natl Acad Sci USA, 1993. 90(8): p. 3725-9.
61. Hamilton, S. E. and J. T. Harty, Quantitation of CD8+ T cell expansion, memory, and protective immunity after immunization with peptide-coated dendritic cells. J Immunol, 2002. 169(9): p. 4936-44.
62. Badovinac, V. P., et al., Accelerated CD8+ T-cell memory and prime-boost response after dendritic-cell vaccination. Nat Med, 2005. 11(7): p. 748-56.
63. Cui, W., et al., Effects of Signal 3 during CD8 T cell priming: Bystander production of IL-12 enhances effector T cell expansion but promotes terminal differentiation. Vaccine, 2009. 27(15): p. 2177-87.
64. Henry, C. J., et al., The roles of IL-12 and IL-23 in CD8+ T cell-mediated immunity against Listeria monocytogenes: Insights from a DC vaccination model. Cell Immunol, 2010. 264(1): p. 23-31.
65. Ahuja, S. S., et al., Dendritic cell (DC)-based anti-infective strategies: DCs engineered to secrete IL-12 are a potent vaccine in a murine model of an intracellular infection. J Immunol, 1999. 163(7): p. 3890-7.
66. Berberich, C., et al., Dendritic cell (DC)-based protection against an intracellular pathogen is dependent upon DC-derived IL-12 and can be induced by molecularly defined antigens. J Immunol, 2003. 170(6): p. 3171-9.
67. Ozawa, Y., et al., Mucosal Vaccine Using Cytotoxic T Cell Epitope-Pulsed Dendritic Cell Confers Protection for Intracellular Pathogen. Am J Respir Cell Mol Biol, 2009.
68. De Vries, I. J., et al., Effective migration of antigen pulsed dendritic cells to lymph nodes in melanoma patients is determined by their maturation state. Cancer Res, 2003. 63(1): p. 12-7.
69. Brockstedt, D. G., et al., Listeria-based cancer vaccines that segregate immunogenicity from toxicity. Proc Natl Acad Sci USA, 2004. 101(38): p. 13832-7.
70. Zitvogel, L., et al., Therapy of murine tumors with tumor peptide-pulsed dendritic cells: dependence on T cells, B7 costimulation, and T helper cell 1-associated cytokines. J Exp Med, 1996. 183(1): p. 87-97.
71. Palucka, A. K., et al., Dendritic cells: a critical player in cancer therapy? J Immunother, 2008. 31(9): p. 793-805.

While the invention has been described in detail and with reference to specific examples thereof, it will be apparent to one skilled in the art that various changes and modifications can be made therein without departing from the spirit and scope thereof.

What is claimed is:

1. An isolated dendritic cell expressing elevated levels of fascin 1, said cell exhibiting accelerated migration.

2. The dendritic cell of claim 1, comprising an expression vector which comprises a nucleic acid encoding fascin 1, operably linked to a constitutive, inducible or dendritic cell-specific promoter.

3. The dendritic cell of claim 1, selected for elevated fascin 1 expression from a population of dentritic cells.

4. A composition comprising isolated dendritic cells as claimed in claim 1 in a biologically acceptable carrier.

5. A method for the treatment of cancer in a patient in need thereof, comprising
a) incubating the cells of a) with a tumor antigen of interest;
b) providing dentritic cells expressing elevated levels of fascin 1;
c) reinfusing the cells of b) into said patient, thereby provoking a cytotoxic T cell response to cancer cells expressing the antigen of step a, said cytotoxic response causing a reduction in tumor cell burden.

6. The method of claim 5, further comprising administration of a chemotherapeutic agent.

7. A method for the treatment of bacterial or viral infection in a patient in need thereof, comprising
a) incubating the cells with a bacterial or viral antigen of interest;
b) providing dentritic cells expressing elevated levels of fascin 1;
c) reinfusing the cells of b) into said patient, thereby provoking a cytotoxic T cell response to infected cells expressing the antigen of step b, said cytotoxic response causing a reduction in infected cell numbers.

8. The method of claim 7, further comprising administration of an antibiotic or anti-viral agent.

9. A method for producing dendritic cells which provoke a cytotoxic T cell response against an antigen of interest comprising;
a) providing the composition of claim 4 comprising said dendritic cells and
b) exposing said cells to an effective concentration of an antigen of interest under conditions effective to prime said cells such that they are effective to induce a cytotoxic T cell response upon reinfusion into a test subject.

10. A method for identifying agents which modulate fascin1 mediated resistance to Listeria monocytogenes (Lm) in dendritic cells, comprising:
a) providing wild type and fascin1 KO DCs infected with Lm;
b) incubating the DCs of step a) in the presence and absence of a test agent;
c) determining replication rate of said Lm in the cells of step b) and identifying agents which differentially alter said rate in fascin 1 KO DCs when compared to wild type DCs.
